(19) United States
(12) Patent Application Publication
(10) Pub. No.: US 2010/0198063 A1
(43) Pub. Date: Aug. 5, 2010

(54) Multi-Modality Phantoms and Methods for Co-registration of Dual PET-Transrectal Ultrasound Prostate Imaging

(75) Inventors: Jennifer S. Huber, El Sobrante, CA (US); William W. Moses, Berkeley, CA (US); Jean Pouliot, Mill Valley, CA (US); I-Chow Hsu, San Francisco, CA (US); Qiyu Peng, Albany, CA (US); Ronald H. Huesman, Danville, CA (US); Thomas F. Budinger, Berkeley, CA (US)

Correspondence Address: LAWRENCE BERKELEY NATIONAL LABORATORY, Technology Transfer & Intellectual Propery Managem, One Cyolotron Road MS 56A-120, BERKELEY, CA, 94720, US

(73) Assignee: THE REGENTS OF THE UNIVERSITY OF CALIFORNIA, Oakland, CA (US)

(21) Appl. No.: 12/622,335

(22) Filed: Nov. 19, 2009

Related U.S. Application Data
(63) Continuation-in-part of application No. PCT/US2008/064160, filed on May 19, 2008.
(60) Provisional application No. 60/939,051, filed on May 19, 2007.

(51) Int. Cl. A61B 8/00; G09B 23/30; G01T 1/164

(52) U.S. Cl. 600/437; 434/267; 250/363.03

(57) ABSTRACT

Herein are described methods and tools for acquiring accurately co-registered PET and TRUS images, as well as the construction and use of PET-TRUS prostate phantoms. Ultrasound imaging with a transrectal probe provides anatomical detail in the prostate region that can be accurately co-registered with the sensitive functional information from the PET imaging. Imaging the prostate with both PET and transrectal ultrasound (TRUS) will help determine the location of any cancer within the prostate region. This dual-modality imaging should help provide better detection and treatment of prostate cancer. Multi-modality phantoms are also described.

### CROSS REFERENCE TO RELATED APPLICATIONS

This application is a continuation-in-part of International Patent Application, PCT/US2008/064160, filed on May 19, 2008, which claims priority to U.S. Provisional Patent Application No. 60/939,051, filed on May 19, 2007, both of which are herein incorporated by reference in their entirety.

### STATEMENT OF GOVERNMENTAL SUPPORT

This invention was made during work supported in part under Contract No. DE-AC02-05CH11231 awarded by the Office of Biological and Environmental Research, Medical Science Division, U.S. Department of Energy, in part under grant numbers DAMD17-02-1-0081 and W81XWH-07-1-0020 awarded by the Department of Defense, in part under grant numbers R01-EB-00194 and R01-HL-071253 awarded by the National Institute for Biomedical Imaging and Bioengineering,. The government has certain rights in this invention.

### BACKGROUND OF THE INVENTION

1. Field of the Invention

The present invention relates to phantoms for use in multi-modality imaging. The present invention specifically relates to positron emission tomograph-ultrasound imaging for prostate cancer detection and therapy.

2. Related Art

More than half of all malignant prostate tumors detected today are not palpable, and prostate specific antigen and digital rectal exam screenings have high false-positive rates in general clinical practice. In addition, the diagnostic accuracy of biopsy is problematic. A new imaging technology is needed for sensitive detection of prostate cancer to confirm diagnosis, guide biopsy and help guide treatment decisions. Administration of multiple courses of a therapy is often needed before a clear indication of response or progression can be determined, so an improved prostate imaging technique is also needed to monitor response to therapy, assess the efficacy of new treatments and detect local recurrence sooner.

We believe that positron emission tomography (PET) can meet many of these needs, because newly developed PET radiopharmaceuticals (such as [¹¹C]choline) have demonstrated outstanding results in the sensitive detection of prostate cancer. Functional PET imaging with [¹¹C]choline detects malignant tumors and helps determine the tumor “aggressiveness,” but few other anatomical features are visible in the PET images.

Prostate cancer imaging with radiopharmaceuticals has used three types of instruments: (1) gamma camera scanning using ^{99m}Tc bone agents, (2) single photon emission tomography (SPECT) using ¹¹¹ In-labeled monoclonal antibodies or ^{99m}Tc-labeled peptides, and (3) positron emission tomography (PET) using [¹⁸F]fluorodeoxyglucose (FDG), [¹¹C]methionine, [¹¹C]choline, [¹¹C]acetate, or [¹⁸F]fluorocholine. ProstaScint™ is an ¹¹¹In-labeled murine monoclonal antibody directed against prostate specific membrane antigen. The acquisition and interpretation of the ProstaScint™ images are technically demanding due to radiopharmaceutical uptake by background organs. The reported sensitivity and specificity of ProstaScint™ imaging for detection of pelvic lymph node metastases is only 62% and 72%, respectively [M. K. Haseman, “Capromab pendetide imaging of occult lymph node metastases,” J Nucl Med, vol. 39, pp. 653, 1998]. Despite these problems, ProstaScint™ imaging is superior to PET imaging with FDG in predicting the presence of prostate cancer [M. K. Haseman, N. L. Reed, and S. A. Rosenthal, “Monoclonal antibody imaging of occult prostate cancer in patients with elevated prostate-specific antigen. Positron emission tomography and biopsy correlation,” Clin Nucl Med, vol. 21, pp. 704-13, 1996], particularly for less aggressive disease [P. F. Faulhaber, D. B. Sodee, E. Echt and J. K. O'Donnell, “Staging of Prostate Adenocarcinoma, comparison of FDG Dedicated PET and In-111 Capromab Pendetide,” J Nucl Med, vol. 41, pp. 116 (abstract), 2000]. While PET has higher sensitivity and spatial resolution than SPECT, FDG is not very prostate specific and bladder accumulation of radioactivity often obscures prostate tumors [E. R. Sigurdson and A. M. Cohen, “Commentary on the applications of PET in clinical oncology,” J. Nucl. Med., vol. 32, pp. 649-650, 1991]. Fortunately, newly developed PET radiopharmaceuticals have recently demonstrated outstanding results in the sensitive detection of prostate cancer. Hara and co-workers find that: [¹¹C]choline clears the blood quickly; its uptake in prostate tumors provides excellent tumor/normal contrast; and bladder accumulation is minimal [T. Hara, N. Kosaka, and H. Kishi, “PET imaging of prostate cancer using carbon-11-choline,” J Nucl Med, vol. 39, pp. 990-5, 1998]. Therefore, [¹¹C]choline is an extremely attractive PET tracer for imaging prostate tumors [See for example, M. Picchio, C. Messa, C. Landoni, L. Gianolli, S. Sironi, et al., “Value of [11C]choline-positron emission tomography for re-staging prostate cancer: a comparison with [18F]fluorodeoxyglucose-positron emission tomography,” J Urol., vol. 169, pp. 1337-1340, 2003]. FIG. 2 shows a [¹¹C]choline image of prostate cancer before and after therapy, demonstrating the ability to detect prostate carcinoma and follow therapy efficacy using [¹¹C]choline. Several other radiopharmaceuticals are also currently under investigation for prostate cancer imaging, including [¹¹C]acetate [E. Fricke, S. Machtens, M. Hofmann, J. Van Den Hoff, S. Bergh, et al., “Positron emission tomography with 11C-acetate and 18F-FDG in prostate cancer patients,” Eur J Nucl Med Mol Imaging, vol. 30, pp. 607-610, 2003], [¹¹C]methionine [ G. Toth, Z. Lengyel, L. Balkay, M. A. Salah, L. Tron, et al., “Detection of prostate cancer with 11C-methionine positron emission tomography,” J. Urol., vol. 173(1), pp. 66-69, 2005], and [¹⁸F]fluorocholine [T. R. Degrado, R. E. Coleman, S. Wang, S. W. Baldwin, M. D. Orr, et al., “Synthesis and Evaluation of F18-labeled Choline as an Oncologic Tracer for Positron Emission Tomography: Initial Findings in Prostate Cancer,” Cancer Research, vol. 61(1), pp. 110-117, 2001, T. R. Degrado, S. W. Baldwin, S. Wang, M. D. Orr, R. P. Liao, et al., “Synthesis and Evaluation of F 18-Labeled Choline Analogs as Oncologic PET Tracers,” J. Nuc. Med., vol. 42, pp. 1805-1814, 2001].

Transrectal ultrasound (TRUS) imaging identifies lesions but some are not cancerous. By accurately co-registering the sensitive functional information from PET imaging with the high resolution anatomical detail from TRUS imaging, dual PET-TRUS prostate imaging can accurately localize prostate cancer. The co-registered PET and TRUS images can then accurately guide subsequent diagnosis and treatment procedures. We discussed the possibility of combining PET and TRUS previously in Huber, J. S.; Moses, W. W.; Pouliot, J.; Hsu, I. C.; Dual-modality PET/ultrasound imaging of the prostate, Nuclear Science Symposium Conference Record, 2005, IEEE, Volume 4, 23-29 Oct. 2005 Page(s):2187-2190, herein incorporated by reference. Herein, we describe the methods and tools for acquiring accurately co-registered PET and TRUS images, as well as the construction and use of PET-TRUS prostate phantoms.

### BRIEF SUMMARY OF THE INVENTION

The present invention provides a method for accurate co-registration for dual-modality positron emission tomography (PET) and transrectal ultrasound (TRUS) imaging for prostate cancer, comprising the steps of: (1) providing a PET-TRUS system comprising a PET scanner having a patient table with a TRUS probe attached to the table through a TRUS calibrated linear stepper-arm assembly (with a holder with 511 keV point sources mounted onto the TRUS stepper); (2) inserting the rectal probe into the patient; (3) acquiring TRUS image data of the prostate stepwise from base to apex; (4) positioning axially the TRUS probe tip at the prostate center using the TRUS stepper; (5) moving the patient bed to position the 511 keV point sources near the PET-center, (6) acquiring PET point source data and analyzing it; (7) moving the patient table to position the prostate of a patient near the PET-center; (8) injecting a 511 keV radiopharmaceutical into the patient; (9) acquiring PET image patient data; (10) 2D contouring the TRUS data and reconstructing a 3D TRUS image of the prostate region; (11) 3D reconstructing the PET data; and (12) accurately superimposing the PET and TRUS images; thus resulting in PET-TRUS images of the patient's prostate region showing anatomical and functional detail for precise localization of any prostate cancer.

In a preferred embodiment, a point source holder, mounted onto the TRUS stepper, places two 511 keV ⁶⁸Ge (<50 μCi) point sources along the axial line of the TRUS probe at a known location from the TRUS probe tip. In a preferred embodiment, the radiopharmaceutical injected in the patient is [¹¹C]choline.

A multi-modality prostate phantom comprising a rigid container comprising a structure simulating an inner cylindrical and spherical prostate region within an outer rectangular pelvic region, said structure comprised of tissue mimicking mixtures and deionized radioactive water. In one embodiment, the tissue mimicking mixture as set forth in Table I.

In another embodiment, the multi-modality prostate phantom structures simulating the rectum, rectal wall and urethra in a background gel with an opening for the TRUS probe. The urethra can be simulated by filling a tube with ultrasound gel and air bubbles.

A multi-modality phantom comprising a shaped gel structure simulating an inner cylindrical and spherical region within an outer rectangular region, said structure comprising tissue mimicking mixtures and radioactive solutions. In one embodiment, the tissue mimicking mixture as set forth in Table 2.

### BRIEF DESCRIPTION OF THE DRAWINGS

FIG. 1. Outline of current and future plans for dual PET-TRUS imaging. We acquire and co-register PET and TRUS images that are taken during the same patient exam. A simulated “merged PET-TRUS image” (shown in the center) represents the goal for each patient imaged. In the future, these co-registered PET and TRUS images will be used to guide subsequent diagnosis and treatment procedures.

FIG. 2. [¹¹C]choline image of prostate cancer before and after treatment. These color images indicate a high (red) uptake in the prostate cancer compared to a low (blue) uptake elsewhere. Images provided by Hara and co-workers [J Nucl Med, vol. 39, pp. 990-5, 1998].

FIG. 3.(a) Photograph of a partially-assembled prostate-optimized PET scanner with the lead shielding on one side removed and a single axial row of detector modules visible. The individual detector modules are angled to point towards the center of the scanner (where the prostate will be positioned). (b) Photograph of the completed scanner with a person in position on the patient table. The detector banks can be tilted to accommodate a patient's bent knees if necessary.

FIG. 4. Transrectal ultrasound system with the probe inserted in a patient, a 2D TRUS image with grid, and contours from a 3D TRUS reconstruction.

FIG. 5.(a) Drawing of a modified TRUS probe-stepper-point source unit. The two point sources on the TRUS stepper are used for patient positioning. All three point sources are used for image co-registration. (b) Photograph of the dual PET-TRUS system including prostate-optimized PET scanner, patient table, TRUS stabilizer arm, TRUS modified stepper, TRUS ultrasound probe, and point source holder (with two ⁶⁸Ge point sources mounted parallel to the TRUS probe axis).

FIG. 6.(a) Ultrasound images of the prostate region taken for brachytherapy treatment planning. (b) 3D view of a prostate (yellow contours), penis bulb (burgundy), bladder (beige), rectum (pink) and urethra (yellow brown). The prostate and urethra contours were measured using TRUS, and the other contours were measured with CT. The turquoise area was defined by MR-spectroscopy as a dominant intraprostatic cancer lesion.

FIG. 7. CIRS Model 58 ultrasound prostate training phantom.

FIG. 8. Design drawing of a realistic custom TRUS-PET prostate phantom. Phantom has structures simulating the prostate, rectal wall and urethra in a background gel with an opening for the TRUS probe.

FIG. 9.(a) Reconstructed image of 37-line source phantom (each 5 cm in axial direction). In the transverse plane, single line sources are 2, 4, 6, and 8 cm from the central line. Clusters of four line sources are placed radially at 4 and 8 cm from the central line. The four line sources in each cluster are spaced 8, 6, 5, and 4 mm apart (clockwise from the 8 mm labeled clusters). Phantom was filled with ¹⁸F at an initial activity of 0.8 mCi and imaged for 2 hours. Phantom was centered in the PET scanner. Image represents 45 M counts. Voxel size equals 2 mm×2 mm×2 mm. (b) Modified NEMA [“Performance Measurements of Positron Emission Tomographs,” National Electrical Manufacturers Association (NEMA) Report No., 2001] body phantom. Six spheres are placed on a 12 cm diameter in the transaxial plane (37, 28, 22, 17, 13, and 10 mm sphere diameters), and a 28 mm diameter sphere is placed in the center. All seven spheres have a common axial center line. (c) Reconstructed images (central slices) of the modified body phantom, which was centered in the PET scanner. Initial ¹⁸F activity density was 1.1 μCi/ml in five spheres (shown in white) and 0.12 μCi/ml in background torso.

FIG. 10.(a) Photograph of a simple PET-ultrasound phantom. The blue-colored agarose has outer dimensions of 16 cm×11 cm×3.5 cm. The yellow-colored gelatin cylinder has a 5 cm diameter. (b) Reconstructed coronal PET image of the PET-ultrasound phantom. The circle shows the high uniform ¹⁸F activity concentration in the prostate region, and the rectangle shows the low uniform ¹⁸F activity concentration in the background pelvis region. Image represents 636 M counts (i.e., 20 minutes of data). The voxel size is 1.47 mm×1.47 mm×3.13 mm. (c) Ultrasound image of the same phantom using an external 5 MHz ultrasound probe. The dark gray circle shows the lower-scatter gelatin prostate, and the surrounding light gray background shows the high-scatter agarose pelvis.

FIG. 11.(a) Photograph of a custom PET-US-CT-MRI phantom. The aqua-colored rectangular pelvis has outer dimensions of 16 cm×11 cm×3.5 cm. The dark green-colored cylindrical prostate has a 5 cm diameter and 2.5 cm depth (i.e. inner cylinder does not go all the way through). (b) Reconstructed coronal PET image of the custom PET-US-CT-MRI phantom. The colored circle shows the ¹⁸F activity in the prostate region; the ¹⁸F activity density was 0.8 μCi/ml in the prostate at the start of the 60 minute emission scan. Voxel size is 3.6 mm×3.6 mm×4 mm. (c) Ultrasound image of the phantom using a 5 MHz external ultrasound probe. The dark gray circle shows the lower-scatter prostate, and the surrounding gray background shows the higher-scatter pelvis. (d) Reconstructed coronal x-ray CT image of the phantom with 1 cm axial thick slices. The pixel size is 1 mm×1 mm×10 mm. The dark gray circle shows lower radiographic attenuation in the prostate compared with the surrounding light gray higher-attenuation pelvis. (e) Reconstructed coronal MRI T₁-weighted image of the custom PET-US-CT-MRI phantom, representing 60 minutes of data. Voxel size is 0.4 mm×0.4 mm×3 mm. The dark gray circle shows the longer T₁ prostate surrounded by the shorter T1 pelvis. (f) Reconstructed coronal MRI T₂-weighted image of the phantom, representing 60 minutes of data. Voxel size is 0.4 mm×0.4 mm×3 mm. (g) Fused PET (color) and TRUS (grayscale) coronal image.

FIG. 12.(a) Reconstructed coronal PET image of a custom PET-TRUS-CTMRI phantom using ⁶⁸Ge. The white circle shows the higher ⁶⁸Ge activity in the “prostate” within the dark gray low ⁶⁸Ge activity in the “pelvis.” The ⁶⁸Ge activity density was 0.73 μCi/ml in the “prostate” and 0.12 μCi/ml in the “pelvis.” Voxel size is 1.47 mm×1.47 mm×3.125 mm. Horizontal profile through the “prostate” center is also shown. (b) Ultrasound image of the phantom. The dark gray circle shows the lower-scatter “prostate,” directly above the black circular hole used for the TRUS probe. The surrounding light gray background shows the higher-scatter “pelvis.”

### DETAILED DESCRIPTION OF THE PREFERRED EMBODIMENT

Multi-modality imaging has an increasing role in the diagnosis and treatment of a large number of diseases, particularly if both functional and anatomical information is acquired and accurately co-registered. Although PET-CT has recently revolutionized the role of imaging in diagnosis and treatment for many kinds of cancer, ultrasound is the preferred imaging technology for many diseases such as prostate cancer. Since transrectal ultrasound imaging is an integral part of diagnosis and treatment for prostate cancer, we describe a dual imaging system that will acquire PET and TRUS data during the same patient imaging session and accurately co-register the images. To validate our methods, we also describe the construction and use of novel custom PET-TRUS prostate phantoms.

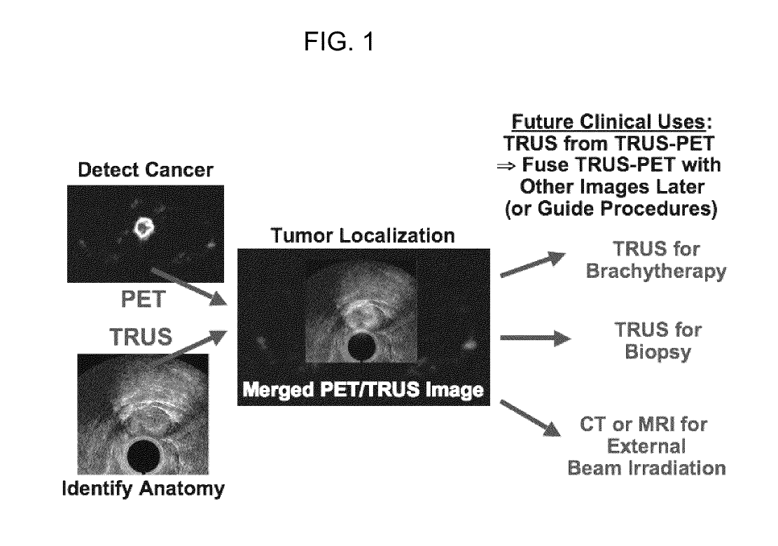

Dual PET-TRUS prostate imaging acquires PET images (to provide sensitive functional information) and transrectal ultrasound images (to give high resolution, 3D volumetric anatomical detail) sequentially during the same patient imaging session, using methods that allow the PET and TRUS images to be accurately co-registered. This combined PET-TRUS system with a single patient table for sequential acquisition of PET and TRUS images helps overcome alignment problems due to internal organ movement, variations in patient table profile, and positioning of the patient for the scan. Dual PET-TRUS imaging will help locate increased metabolic activity (i.e., cancer) within the prostate region. This dual prostate imaging should help confirm initial diagnosis, guide biopsy, guide treatment decisions, monitor response to therapy, and detect local reoccurrence. Ultimately it should provide better detection and treatment of prostate cancer. FIG. 1 shows the general motivation behind dual PET-TRUS imaging. A simulated “merged PET-TRUS image” (shown in the center) represents the goal for each patient imaged. FIG. 1 also outlines some possible applications for dual PET-TRUS imaging. Dual positron emission tomography and transrectal ultrasound prostate imaging has not been done previously.

PET is fundamentally different than most imaging technologies commonly used for prostate cancer detection, because PET is based on function (e.g., whether or not a radiopharmaceutical is taken up by the prostate tumors) rather than anatomical structure (e.g., whether or not a tumor mass is observed using X-ray computed tomography or transrectal ultrasound). Our dual PET-TRUS prostate imaging tools and methods may be applied to whole-body PET scanners using positron-emitting tracers, including but not limited to, PET radiopharmaceuticals such as, [¹¹C]choline, [¹⁸F]fluorodeoxyglucose (FDG), [¹¹C]methionine, [¹¹C]choline, [¹¹C]acetate, or [¹⁸F]fluorocholine.

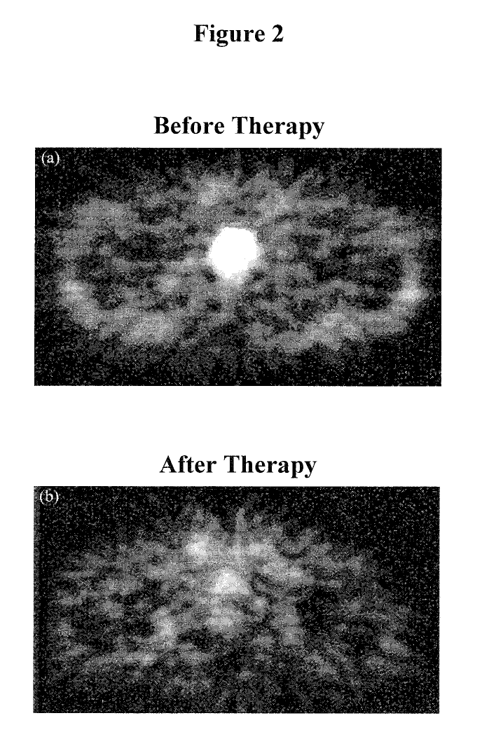

PET imaging is especially useful for prostate imaging because functional PET imaging will detect malignant tumors in the prostate region, as well as help determine tumor “aggressiveness” based on metabolic uptake levels. Although some scanners are not optimized to detect distant metastatic disease, in a preferred embodiment, the PET scanner used should also be able to image local spread beyond the prostate bed. FIG. 2 shows a [¹¹C]choline image of prostate cancer before and after therapy, demonstrating the ability to detect prostate carcinoma and follow therapy efficacy using choline.

However, FIG. 2 also shows that the relative uptake in a tumor can be so great that few other anatomical landmarks are visible in the PET images. PET imaging is therefore greatly enhanced if its functional information is fused with anatomical information. Transrectal ultrasound imaging provides high resolution anatomical detail in the prostate region that can be co-registered with the sensitive functional information from PET imaging. TRUS imaging can also be used to help align the patient (with the prostate near the center of the PET scanner), which is important due to the limited axial extent of some PET scanners. Transrectal ultrasound imaging of the prostate is a standard imaging technique widely used for prostate cancer diagnosis, biopsy, treatment planning and brachytherapy seed placement. A TRUS probe is relatively small and the procedure is generally well tolerated by patients. We have chosen transrectal ultrasound over CT to provide localization and anatomical imaging because TRUS is cheaper, does not expose the patient to radiation, and provides outstanding prostate image quality.

It is important to acquire the PET and TRUS data sets during the same imaging session. If PET and TRUS images are taken independently (at different times), the co-registration of the images needs to be based on image features such as body contour. This registration method is difficult and inaccurate, since PET images do not show detailed anatomical features. Acquiring the PET and TRUS prostate images during the same exam is necessary for the following main reasons: (1) The TRUS probe distorts the soft-tissue anatomy, so the rectal probe needs to be in place during the PET scan; (2) PET prostate images do not show detailed anatomical landmarks and the TRUS probe position is patient dependent, so the position of the TRUS probe (relative to the center of the PET scanner) needs to be determined for each patient; (3) PET scanners have a limited axial extent, so image resolution may be improved if transrectal ultrasound is used to help locate and position the patient's prostate near the center of the PET scanner; (4) It is easier for the patient to have both procedures done together in one appointment. In short, patient positioning and accurate image co-registration require the PET and TRUS images to be sequentially acquired during the same imaging session.

In order to take full advantage of new prostate cancer radiopharmaceuticals, in a preferred embodiment, a prostate-optimized PET scanner is used to image the prostate, such as the high performance positron emission tomograph scanner optimized to image the prostate and described in J. S. Huber, S. E. Derenzo, J. Qi, W. W. Moses, R. H. Huesman, et al., “Conceptual Design of a Compact Positron Tomograph for Prostate Imaging,” IEEE Trans Nucl Sci, vol. NS-48, pp. 1506-1511, 2001 and J. S. Huber, W. S. Choong, W. W. Moses, J. Qi, J. Hu, et al., “Initial Results of a Positron Tomograph for Prostate Imaging,” IEEE Trans Nucl Sci, NS-53, pp. 2653-2659 (2006), hereby incorporated by reference and also described in Example 1. Compared with a standard whole-body PET scanner (e.g., the CTI EXACT HR or HR+ and GE Advance), a prostate-optimized PET scanner has comparable sensitivity and resolution, less background, and a lower cost. Performance of the prostate-optimized PET scanner is described in detail in a recent paper, J. S. Huber, W. S. Choong, W. W. Moses, J. Qi, J. Hu, G. C. Wang, D. Wilson, S. Oh, R. H. Huesman, S. E. Derenzo, and T. F. Budinger, “Initial Results of a Positron Tomograph for Prostate Imaging, ” IEEE Trans. Nucl. Sci, NS-53, pp. 2653-2659, 2006, herein incorporated by reference.

In other preferred embodiments, known and commercially available PET or PET/CT scanners can be used to carry out dual modality PET-TRUS prostate imaging. Such scanners include but are not limited to those commercially sold by Siemens (e.g., Biograph, ECAT ACCEL, or ECAT EXACT HR+), GE Healthcare (e.g., Discovery PET/CT), and Philips (e.g., CPET, Gemini, or Allegro).

The transrectal ultrasound imaging probe used for the dual PET-TRUS imaging can be acquired commercially. Some commonly used TRUS probes include Hitachi Medical Systems EUP-U533, Aloka UST-672-5/7.5 and UST-678, and B-K Medical model 8808 and 8658. In one embodiment, the transrectal ultrasound imaging system is a commercially available system, such as the Hitachi Hi Vision 5500. In another embodiment, the TRUS stepper and stabilizer arm assembly is a commercially available stepper and arm assembly, such as the Accucare EXII stepper with micro-touch stabilizer arm. However, modifications to the TRUS stepper-arm assembly are likely required to work in conjunction with a PET scanner and would be appreciated by one having skill in the art.

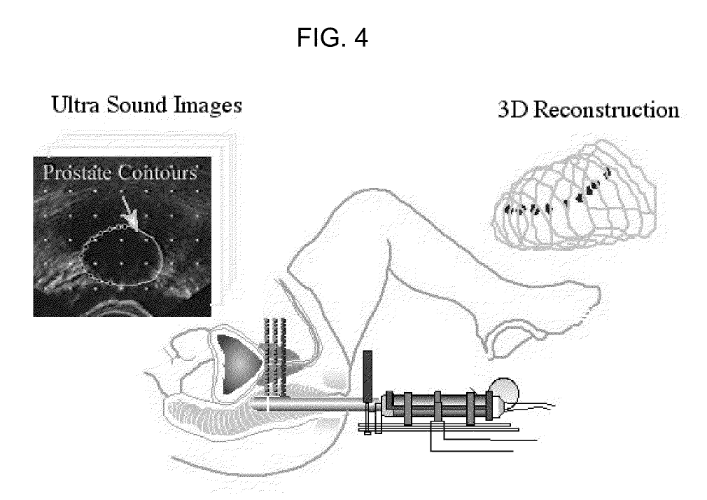

We first describe a method for positioning a patient's prostate near the center of the PET scanner (“PET-center”). As used herein, by the term, “PET-center,” it is meant the center of the imaging volume of a PET scanner. This prostate positioning is particularly important for the prostate-optimized PET scanner, since it has a limited axial extent of 8 cm. In a preferred embodiment, the transrectal ultrasound probe will be rigidly attached to a calibrated linear stepper that allows linear displacement along its axis, as shown in FIG. 4. In one embodiment, the calibrated linear stepper that allows displacement along its axis is motorized and the stepper is controlled by computer means. In another embodiment, the linear stepper is controlled manually. In a preferred embodiment, the stepper also has calibrated control (manual or motorized) of the TRUS probe rotational motion. The TRUS equipment is commercially available and may be used in the present invention. For example, the TRUS system used in the present Examples is a Hitachi Hi Vision 5500 with a EUP-U533 bi-plane transrectal transducer probe, Accucare EXII stepper, and micro-touch stabilizer arm, or equivalent.

The TRUS probe is rigidly mounted in a TRUS calibrated linear stepper. This TRUS probe-stepper unit is mounted onto a moveable TRUS stabilizer arm that is rigidly attached to the patient table. The arm moves to allow correct positioning of the TRUS probe in a patient or phantom, then its position is fixed typically using the locking mechanism(s) provided with the commercial TRUS arm or through a fastening means. In a preferred embodiment, the TRUS stabilizer arm position is fixed by tightening a single knob locking mechanism. The patient table is equipped with means of supporting the patient's torso, abdomen, legs and/or feet to prevent movement after the probe has been inserted and positioned.

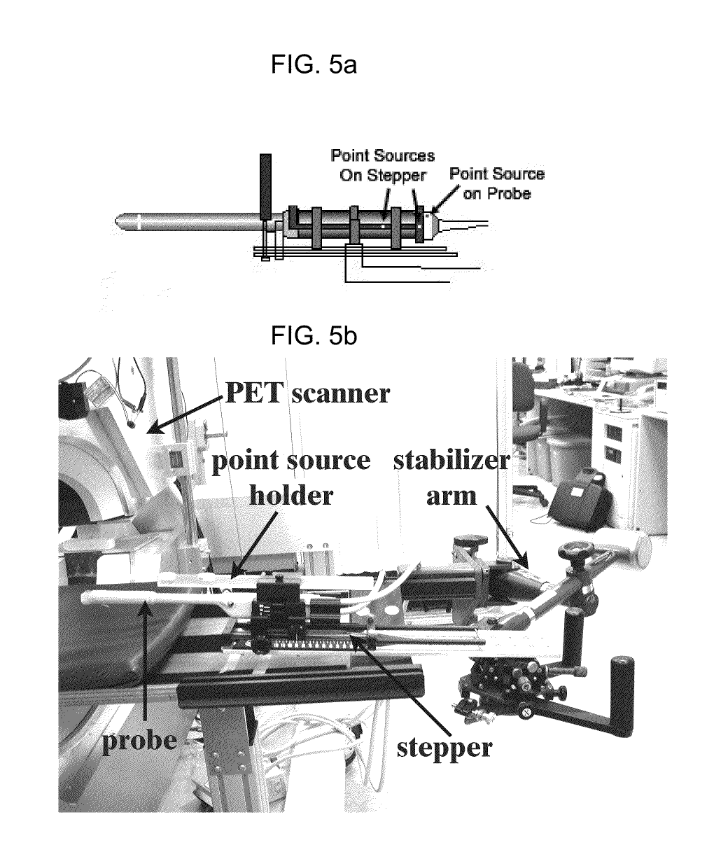

In one embodiment, three point sources are used to determine the TRUS probe location relative to the PET-center. Two 511 keV PET point sources are placed in a holder that is attached to the TRUS stepper. The two point sources are accurately placed along the axial line of the TRUS probe at a known location from the TRUS probe tip (FIG. 5a). Only these two point sources are needed for prostate positioning. In a preferred embodiment, the point sources are <50 μCi ⁶⁸Ge point sources. A third non-collinear 511 keV PET point source is placed on the rear of the probe to define the probe rotation, and all three point source locations are used for image co-registration. The point sources can be purchased from a commercial vendor (e.g., Isotope Products Laboratories model MMS01).

In another embodiment, an alternate technique is to mechanically restrict one or more dimensions of the TRUS probe-stepper assembly after the probe is inserted into the patient, thus eliminating the need for one of the PET point sources during patient studies. For instance, in the preferred embodiment, the rotation of the TRUS probe is always set to the specific angle of zero rotation using the calibrated rotational control of the TRUS stepper (which is available in addition to the linear stepper control); thus eliminating the need for the third point source on the rear of the TRUS probe.

Another alternate technique would be to develop a TRUS probe assembly that utilizes an articulated arm with multiple angular encoders on it, allowing the absolute spatial resolution of the ultrasound images to be known. This arm would also be used to identify the PET-center in the same coordinates.

In a preferred embodiment, the TRUS probe is attached to a calibrated stepper and a series of 2D TRUS images in the transverse plane (i.e., perpendicular to the probe axis) are taken as the probe is moved in steps past the prostate. The TRUS images are acquired every 2-5 mm from base to apex. The 2D images are then reconstructed to visualize a single 3D volumetric image of the prostate, urethra and rectum wall. Such 3D TRUS images are currently used to determine the prostate volume and calculate dose for brachytherapy planning. In one embodiment, a physical puncture attachment for radiotherapeutic seed implantation is also attached to the TRUS stepper. A virtual grid position, corresponding to the projection of the puncture attachment holes, is projected on the TRUS image to provide localization. FIG. 4 shows a drawing of the TRUS unit with the probe inserted in a patient, as well as a 2D transverse ultrasound image with grid and the contours from a 3D reconstruction. In one embodiment, the probe is controlled step-wise using the motorized and computer means. The movement of the probe from base to apex of the prostate can be automated to occur in stepwise distance and timed increments coinciding with the acquisition of a 2D ultrasound image. It is estimated that a brachytherapist or skilled or trained technician should be able to position the probe in the patient in about 2 minutes, and acquire 10-30 2D ultrasound images (or “slices”) in about 3 minutes. No anesthesia is required.

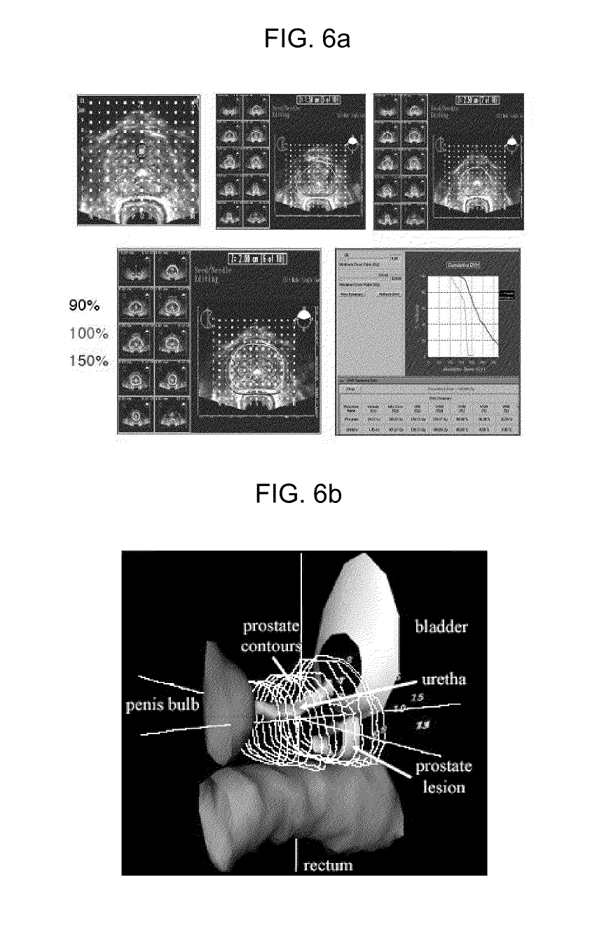

The present TRUS system should provide 3D volumetric images of the prostate. FIG. 6a shows TRUS images of the prostate region which were taken for brachytherapy planning to determine the optimal dose and seed distribution. The top left image shows the axial view of an ultrasound image of the prostate, urethra and anterior section of the rectum.

The top center and right images show the prostate and urethra which are contoured on each plane from base to apex. The bottom plots show the dose distribution superimposed on an ultrasound image to present the dose delivered to the prostate. FIG. 6b shows a 3D reconstructed TRUS image. The prostate and urethra contours were reconstructed from TRUS transverse contours, and the other contours were measured using CT.

In another embodiment, the longitudinal transducer on the TRUS probe is used instead of the transverse transducer. The stepper may have two modes: (1) step linearly along the probe axis or (2) step rotationally around the probe axis. In this case, the TRUS probe is not stepped in the linear direction. Instead, the biplane transrectal ultrasound probe is positioned (to view the entire length of the prostate at once) and rotated in steps, in order to acquire TRUS image data for the entire prostate region. Contouring can still be performed to create a 3D volumetric TRUS image.

Once the TRUS probe is inserted and positioned in the patient (or phantom), the stabilizer arm position is fixed, and the TRUS image data are acquired, then the TRUS probe tip is positioned axially at the center of the prostate using the linear stepper. The patient table is then moved so the point sources are visually positioned near the PET-center with the aid of visible low-powered lasers mounted on the gantry along the minor and major axis of the PET scanner. PET point source data are then acquired for 1-5 minutes, analyzed, and the location of the point sources determined in PET coordinates (i.e., relative to the PET-center). In a preferred embodiment, the data analysis uses a two-iteration expectation-maximization algorithm, with a simplified model of the PET scanner geometry, to quickly reconstruct the data in less than 5 minutes. The resulting PET images are then quickly processed to determine the point source locations in PET coordinates, by determining the brightness-weighted average for each point source in the 3D volumetric PET images. In another embodiment, the data analysis is performed by rebinning the data into a single 2D sinogram, and determining the point source locations using the sinogram data; no image reconstruction of the point sources is needed for patient positioning for this embodiment.

Once the point source locations are determined in PET coordinates, these locations are represented by two position vectors and vector algebra is used to calculate the location of the TRUS probe tip. Finally, the patient table is then moved to position the TRUS probe tip (i.e., prostate) at the PET-center and patient (or phantom) PET data is acquired. After the prostate is positioned, the point sources can be removed for the remainder of the study or they can be left in place (since they are outside the PET imaging volume and have such low activity that patient dose is trivial).

Thus, in the general method, the probe is inserted into the patient, the TRUS imaging is done, the patient table is moved to acquire and analyze point source PET data, the patient table is moved to locate the patient's prostate near the PET-center, then PET imaging of the patient is performed. In a preferred embodiment, a method for accurate co-registration for dual-modality positron emission tomography and transrectal ultrasound imaging for prostate cancer, comprises the steps of: providing a PET-TRUS system comprising a PET scanner having a patient table with a TRUS probe attached to the table through a TRUS calibrated linear stepper-arm assembly (with a holder with 511 keV point sources mounted onto the TRUS stepper); inserting the rectal probe into the patient; acquiring TRUS image data of the prostate stepwise from base to apex; positioning axially the TRUS probe tip at the prostate center using the TRUS stepper; moving the patient bed to position the 511 keV point sources near the PET-center, acquiring PET data and analyzing it; moving the patient table to position a patient's prostate near the PET-center; injecting a 511 keV PET radiopharmaceutical into the patient; acquiring PET image patient data; 2D contouring the TRUS data and reconstructing a 3D TRUS image of the prostate region; 3D reconstructing the PET data; and accurately superimposing the PET and TRUS images; thus resulting in PET-TRUS images of the patient's prostate region showing anatomical and functional detail for precise localization of any prostate cancer.

The TRUS probe will remain in the patient during the PET imaging to minimize organ motion. The TRUS prostate images will be exported (e.g. in BMP or TIFF format) to one of many different platforms to create contours such as in FIG. 4. In one embodiment, the contours are specified by points in xyz coordinate space, and a simple text file is exported to the PET reconstruction computer where the TRUS prostate contours are superimposed with the PET prostate images using methods and software described in and adapted from G. J. Klein, X. Teng, W. J. Jagust, J. L. Eberling, A. Acharya, et al., “A methodology for specifying PET VOI's using multimodality techniques,” IEEE Trans Med Imag, vol. 16, pp. 405-415, 1997, and R. H. Huesman, G. J. Klein, J. A. Kimdon, C. Kuo and S. Majumdar, “Deformable registration of multimodal data including rigid structures,” IEEE Trans Nucl Sci, vol. 50, pp. 389-392, 2003, both of which are hereby incorporated by reference.

In another embodiment, the PET prostate images can be combined with the TRUS reconstructed image and can be presented in a variety of ways, including 2D TRUS contours, 3D TRUS contours, or the TRUS images superimposed with the PET images. A large number of software packages or custom software can be used for image fusion, allowing the simultaneous display of PET images, ultrasound images and contours. Such commercial platforms to perform image fusion between any 2 DICOM format 3D image sets include but are not limited to Occentra-MasterPlan™, Nucletron, RTT Coherence™ and Leonardo™ Workstation, Siemens and also Pinnacle™ (Phillips). These applications can also display simultaneously 2D contours and points. We have also developed application tools (using Matlab and DICOM-convert) to translate any 3D data set from raw or other known format (Tiff, jpeg, etc.) into a DICOM format.

The ability to position the prostate can be validated by imaging a point source that represents the prostate location. One embodiment is to verify prostate positioning by attaching a third 511 keV ⁶⁸Ge point source on the TRUS probe tip and positioning the probe tip at the PET-center using the previously described procedure. The positioning error is then determined by comparing the third point source location (from a 3D PET image) to its expected location (e.g., the PET-center). PET data are acquired, a 3D PET image of the point source at the probe tip is reconstructed, and the third point source location in PET coordinates is determined. This procedure is repeated several times. Although the spatial resolution of the PET scanner is 4 mm FWHM, the centroid of the profile can be more accurately determined The TRUS probe tip should be positioned reproducibly (e.g., within 1 mm) from the PET-center. A patient's prostate only needs to be positioned within the optimum central field of view of the scanner near the PET-center. In a preferred embodiment, when using an prostate-optimized PET scanner, the patient's prostate should be positioned within 3 cm from the PET-center in the transaxial plane and 1 cm from the PET-center in the axial plane. In another embodiment, the patient's prostate is positioned to within at 4 mm from the PET-center.

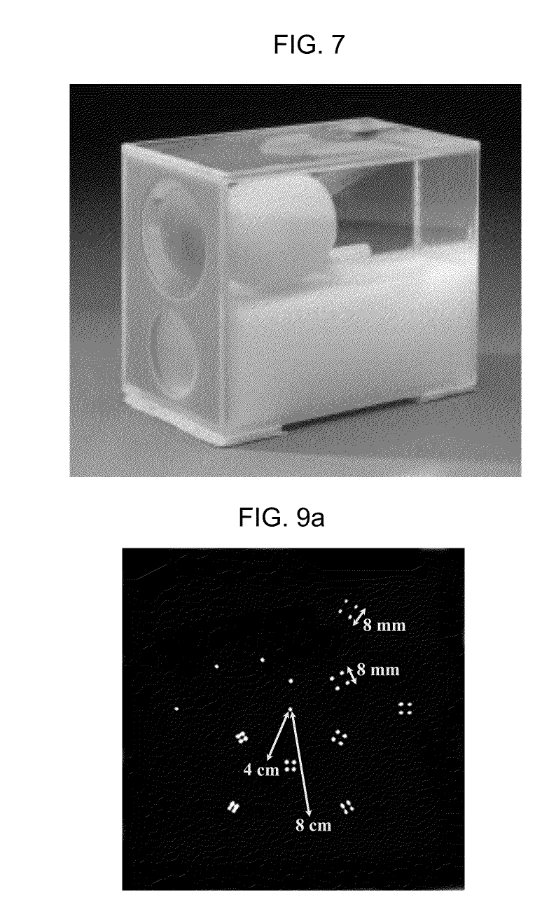

In another embodiment, prostate positioning is validated using a 511 keV ⁶⁸Ge point source that is placed at a fixed known position on the prostate in a commercial TRUS prostate phantom (e.g., CIRS model 058), which is a clear acrylic box with structures simulating the prostate, rectal wall, seminal vesicles, urethra and perineal membrane (FIG. 7). In another embodiment, validation is performed with a commercial TRUS prostate phantom that has tubing filled with a 511 keV radioactive solution (e.g., ¹⁸F-water). The tubing is placed at a fixed known location through the prostate of the TRUS phantom. For either embodiment, the phantom is placed on the patient table and imaged with the TRUS probe in 2-5 mm steps from base to apex of the prostate. The patient table is then moved to center the prostate at the PET-center (using the previously described procedure), PET phantom data is acquire and reconstructed. If the PET images indicate that the PET (point or line) source is near the PET-center, then prostate positioning is successfully validated. This can be further validated by repeating the phantom imaging procedure several times to confirm that the prostate positioning is reproducible.

The ability to accurately co-register PET and TRUS images can be validated by constructing and imaging a custom PET-TRUS prostate phantom. Methods on ultrasound phantom construction are described in literature [See W. D. D'Souza, E. L. Madsen, O. Unal, K. V. Vigen, G. R. Frank, et al., “Tissue mimicking materials for a multi-imaging modality prostate phantom,” Med. Phys., vol. 28, pp. 688-700, 2001; K. J. M. Surry, H. J. B. Austin, A. Fenster and T. M. Peters, “Poly(vinyl alcohol) cryogel phantoms for use in ultrasound and MR imaging,” Phys Med Biol, vol. 49, pp. 5529-5546, 2004; and E. L. Madsen, M. A. Hobson, S. Hairong, T. Varghese and G. R. Frank, “Tissue-mimicking agar/gelatin material for use in heterogeneous elastography phantoms,” Phys. Med. Biol., vol. 50, pp. 5597-5618, 2005] with the exception of any discussion of the manufacturing of PET-ultrasound phantoms. The custom PET-TRUS prostate phantom has structures that simulate the acoustical properties for TRUS imaging and 511 keV activity concentrations for PET imaging. In one embodiment, the PET-TRUS phantom can be made of agar-gelatin-based tissue mimicking materials (TMMs) that are mixed with radioactive water solutions. The TMMs can be made compatible with MR imaging through the correct choice of materials. Since most commercial PET scanners now have CT capability, the phantom can also be made CT compatible (e.g., by adding concentrations of iodine contrast agent or barium sulfate to the radioactive water solutions).

A PET-TRUS phantom can be constructed using short-lived radioactivity, such as ¹⁸F (110 minutes half-life). Short-lived radioactivity is readily available from in-house cyclotrons or commercial companies that deliver 18F-fluorodeoxyglucose. If long term repeated use of the phantom is desired, then the phantom needs to be constructed with a long-lived radioactivity, such as ⁶⁸Ge radioactivity (271 days half-life).

In one embodiment, a PET-TRUS prostate phantom with a simple geometry is used for validation. In one embodiment, the multi-modality prostate phantom comprising a rigid container comprising a structure simulating an inner cylindrical prostate region within an outer rectangular pelvic region. The phantom is comprised of a cylinder or spherical prostate with 511 keV radioactivity concentrated uniformly, and an outer background pelvis with a different uniform concentration of 511 keV radioactivity. For instance the 511 keV activity density could be three times higher in the prostate compared to the pelvis.

The phantom can be constructed with ultrasound agar-gelatin-based TMMs with different ultrasound scatter properties for the prostate and pelvis, using a tissue-mimicking mixture. Similar agar-gelatin mixtures were proven to have long-term stability at room temperature for at least one year by Madsen, et al. [E. L. Madsen, M. A. Hobson, S. Hairong, T. Varghese and G. R. Frank, “Tissue-mimicking agar/gelatin material for use in heterogeneous elastography phantoms,” Phys. Med. Biol., vol. 50, pp. 5597-5618, 2005]. In one embodiment, the structure simulating an inner cylindrical or spherical prostate region within an outer rectangular pelvic region is comprised of tissue mimicking mixtures of agar, gelatin, CuCl₂-2H₂O, EDTA-tetra Na Hydrate, NaCl, HCHO, anti-bacterial and/or anti-fungal preservative, glass beads, BaSO₄, and deionized radioactive water as set forth in Table I. [J. S. Huber, Q. Peng, and W. W. Moses, “Multi-Modality Phantom Development,” IEEE Nuclear Science Symposium Conference Record 2007, vol. 4, pp. 2944-2948, (Edited by B. Yu), Honolulu, Hi., 2007].

The simple phantom is produced in two stages. First the outer pelvis is filled, creating an inclusion with a petrolatum-coated rod in the center. This rod is removed, then the inner prostate is filled with a TMM with different acoustical properties and activity concentration. Similarly, a second rod can be used to create an inclusion for the TRUS probe to allow TRUS imaging. In a preferred embodiment, a membrane-sealed hole is created in the radioactive pelvis gel for the TRUS probe.

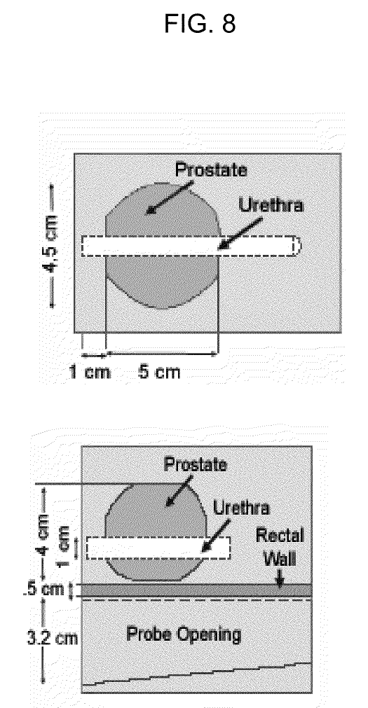

In a preferred embodiment, a TRUS-PET prostate phantom with realistic anatomy can be used for validation. For instance, the phantom having structures simulating the prostate, rectal wall and urethra in a background gel with an opening for the TRUS probe, as shown in FIG. 8. If this TRUS-PET prostate phantom is only used to validate image co-registration, the phantom does not have to exactly mimic tissue or anatomy of the pelvis region. It can be constructed using a variety of tissue mimicking materials, such as the one described above and shown in FIG. 8.

In another embodiment, tissue mimicking materials could be used other than agar-gelatin mixtures. Typical TMMs include agar, Zerdine™, urethanes, epoxies, liquids and natural materials. There are three TMMs commercially available: Zerdine™ from CIRS Inc., condensed-milk-based gel from Gammax RMI, and urethane-rubber-based material from ATS Labs. Alternative phantom construction using radioactive water in condensed milk-agar-based mixtures [W. D. D'Souza, E. L. Madsen, O. Unal, K. V. Vigen, G. R. Frank, et al., “Tissue mimicking materials for a multi-imaging modality prostate phantom,” Med. Phys., vol. 28, pp. 688-700, 2001] or polyvinyl alcohol) cryogels [K. J. M. Surry, H. J. B. Austin, A. Fenster and T. M. Peters, “Poly(vinyl alcohol) cryogel phantoms for use in ultrasound and MR imaging,” Phys Med Biol, vol. 49, pp. 5529-5546, 2004] can be used. The urethra could also be simulated by filling a tube with ultrasound gel with some air bubbles.

In another embodiment, image co-registration can be validated using a PET-TRUS phantom similar to the methods described above. Namely, tubing is added through a commercial TRUS prostate phantom (such as CIRS model 058) and filled with 511 keV radioactive solution. The tubing is placed at a fixed known location through the prostate of the TRUS phantom. The tubing material is chosen for clear ultrasound imaging (e.g., the entire tube outline cross-section should be visible in coronal ultrasound images) and appropriate dimensions (e.g., large enough inner volume for the necessary 511 keV radioactive solution).

In order to validate our co-registration methods, the custom PET-TRUS prostate phantom is placed on the patient table, TRUS and PET prostate images are acquired and reconstructed, and the images co-registered (using the method described above). The dual-modality imaging phantom and patient data can be presented in a variety of ways, including 2D TRUS contours, 3D TRUS contours, or the TRUS images superimposed with the PET images. A large number of software packages or custom software can be used for image fusion, allowing the simultaneous display of PET images, ultrasound images and contours.

In a preferred embodiment, a prostate-optimized PET scanner is used and PET images are reconstructed using a 3D iterative penalized maximum likelihood algorithm as described in J. S. Huber, S. E. Derenzo, J. Qi, W. W. Moses, R. H. Huesman, et al., “Conceptual Design of a Compact Positron Tomograph for Prostate Imaging,” IEEE Trans Nucl Sci, vol. NS-48, pp. 1506-1511, 2001; R. H. Huesman, G. J. Klein, W. W. Moses, J. Qi, B. W. Reutter, et al., “List mode maximum likelihood reconstruction applied to positron emission mammography with irregular sampling.,” IEEE Trans Med Imag, vol. 19, pp. 532-537, 2000; and J. Hu, J. Qi, J. S. Huber, W. W. Moses and R. H. Huesman, “MAP image reconstruction for arbitrary geometry PET systems with application to a prostate-specific scanner.” Proceedings of The International Meeting on Fully Three-Dimensional Image Reconstruction in Radiology and Nuclear Medicine, pp. 416-420, Salt Lake City, Utah, 2005, hereby incorporated by reference (although use of other reconstruction algorithms are also possible). The attenuation correction factors are calculated based on body contours and a uniform attenuation coefficient. Anatomical boundaries are obtained from the outer edges of emission sinograms acquired from transverse sections [C. Michel, A. Bol, A. G. DeVolder and A. M. Goffinet, “Online brain attenuation correction in PET: towards a fully automated data handling in a clinical environment,” Euro J Nucl Med, vol. 15, pp. 712-718, 1989]. Heterogeneous attenuation coefficients could also be used, by identifying tissue types (e.g., soft tissue, bone, and air) based on the TRUS ultrasound images. In another embodiment, a commercially available PET scanner is used with the provided reconstruction software, and transmission scan data is typically used for attenuation correction. In either embodiment, attenuation correction is made for the TRUS probe (which is left in place at a known location during the PET scan) and the patient table.

In another embodiment, further validation of the co-registration of these images is made by taking transversal and sagittal TRUS images during the PET procedure to measure the extent of prostate motion during PET imaging. Motion correction algorithms can be used on the patient PET data if necessary [See G. J. Klein, B. W. Reutter, and R. H. Huesman, “Four-dimensional affine registration models for respiratory-gated PET,” IEEE TNS Nucl Sci, vol. 48, pp. 756-760, 2001, and G. J. Klein and R. H. Huesman, “Four-dimensional processing of deformable cardiac PET data,” Med Imag Anal, vol. 6, pp. 29-46, 2002]. As the data can be acquired in listmode, they lend themselves to retrospective motion correction.

In another embodiment, dual PET-TRUS can be validated by initial patient studies. Radiopharmaceutical uptake in a patient's prostate should be visible with the PET scanner after injection if the patient is positioned properly. The patient positioning technique is successfully validated if the prostate has been positioned within the optimum central field of view of the PET scanner as evidenced by PET imaging. If no radiopharmaceutical uptake is seen in the field of view of the PET scanner, then there is some uncertainty. This patient may not have enhanced radiopharmaceutical uptake in his prostate or he may not have been properly positioned in the PET scanner. PET data can be acquired at the neighboring regions to eliminate potential uncertainty. For instance, the patient table could be moved ±7 cm from the initial position and PET data acquired for at each position. If the patient has no enhanced radiopharmaceutical uptake in either of these neighboring regions, then the patient positioning is considered valid.

In one embodiment, once co-registered PET and TRUS images have been acquired and have localized prostate cancerous tumors, these PET and TRUS images can accurately guide subsequent diagnosis and treatment procedures. For instance, dual PET-TRUS prostate imaging could be used to guide biopsy. Clinical staging and treatment decisions are largely based on biopsy confirmation of prostate cancer, with the Gleason histologic grading recognized as the best indicator of prognosis currently available. However, the diagnostic accuracy of biopsy is problematic, mainly due to sampling effects caused by tumor heterogeneity and to interpretational bias [D. Gleason, “Histologic grading of prostate cancer: A perspective,” Hum Pathol, vol. 23, pp. 273-279, 1992]. There is up to a 1-in-3 chance that the underlying pathologic Gleason grade is higher than the Gleason grading from sextant prostate biopsies [C. R. King, J. E. McNeal, H. Gill and J. C. Presti, “Extended Prostate Biopsy Scheme Improves Reliability of Gleason Grading: Implications For Radiotherapy Patients,” Int. J. Radiation Oncology Biol. Phys., vol. 59, pp. 386-391, 2004]. For the sub-population of men with rising PSA levels and non-diagnostic biopsies or TRUS techniques, dual PET-TRUS prostate imaging could help target the area to biopsy. It has been shown that a 10-core peripheral zone biopsy scheme (compared to sextant biopsies) improves the Gleason grade reliability. An even higher degree of sampling with image-directed cores could yield even better agreement between biopsy and surgical grade, carrying important implications for management decisions [C. R. King, J. E. McNeal, H. Gill and J. C. Presti, “Extended Prostate Biopsy Scheme Improves Reliability of Gleason Grading: Implications For Radiotherapy Patients,” Int. J. Radiation Oncology Biol. Phys., vol. 59, pp. 386-391, 2004]. Biopsy is used for initial diagnosis, guiding treatment decisions, and detecting local recurrence after therapy. Using dual PET-TRUS prostate imaging to increase the diagnostic accuracy of biopsy could have a major clinical impact at several stages of disease management.

In another embodiment, dual PET-TRUS prostate imaging could be used to guide treatment decisions. For example, dual PET-TRUS prostate imaging could aid in treatment planning of external beam irradiation and brachytherapy. Dual PET-TRUS prostate imaging should help determine which part of the prostate and/or prostate table needs higher treatment dose by identifying the location and aggressiveness of the prostate cancer. This improvement could reduce the dosage to surrounding normal tissue, which could reduce treatment side effects.

In another embodiment, dual PET-TRUS prostate imaging could be used to monitor response to therapy. Current means of assessing treatment response in prostate cancer are imprecise because changes in tumor size may be difficult to document, and changes in serum PSA do not always correlate well with clinical outcomes. Transrectal ultrasound is used to identify lesions but some of the detected lesions are not cancerous, so transrectal guided biopsy is needed to follow a patient. The administration of multiple courses of a therapy is often necessary before a clear indication of response or progression can be determined For the sub-population of patients post-therapy with an increasing PSA level and no definitive recurrence evident, dual PET-TRUS prostate imaging could detect an early failure response to therapy. Currently, it typically takes six months to a year to determine treatment failure, largely due to the high false-positive rate of PSA testing, and approximately 20-50% of patients have local recurrence (J. A. Connolly, K. Shinohara, J. C. Presti and P. R. Carroll, “Local Recurrence after Radical Prostatectomy: Characteristics In Size, Location, and Relationship to Prostate-Specific Antigen and Surgical Margins,” Urology, vol. 47, pp. 225-231, 1996; S. E. Lerner, M. L. Blute, E. J. Bergstralh, D. G. Bostwick, J. T. Eickholt, et al., “Analysis of risk factors for progression in patients with pathologically confined prostate cancers after radical retropubic prostatectomy,” J Urol., vol. 156, pp. 137-143, 1996; D. A. Kuban, H. D. Thames, L. B. Levy and et. al., “Long-term multi-institutional analysis of stage T1-T2 prostate cancer treated with radiotherapy in the PSA era,” Int J Radiat Oncol Biol Phys, vol. 57, pp. 915-928, 2003; J. Crook, S. Malone, G. Perry, Y. Bahadur, S. Robertson, et al., “Postradiotherapy prostate biopsies: What do they really mean? Results for 498 patients.,” Int J Radiat Oncol Biol Phys, vol. 48, pp. 355-367, 2000; and A. L. Zietman, M. L. DeSilvio, J. D. Slater and et. al., “Comparison of conventional-dose vs. high-dose conformal radiation therapy in clinically localized adenocarcinoma of the prostate: a randomized controlled trial.,” Jama., vol. 294, pp. 1233-1239, 2005). PET imaging with radiopharmaceuticals, such as [¹¹C]choline, has the potential to effectively detect prostate cancer about a month after therapy once the initial healing has occurred. This represents a large potential improvement in therapy monitoring that would have a significant clinical impact

In another embodiment, the TRUS images (from dual PET-TRUS) can be used to accurately co-register the PET images (from dual PET-TRUS) to other high-resolution anatomical images of the prostate, such as CT, MRI, or TRUS images taken on different days. This would provide improved subsequent co-registration with the PET images than if the PET images were co-registered directly, since PET images of the prostate do not contain high-resolution anatomical detail. TRUS images from a biopsy, volume study, and brachytherapy seed placement are routinely co-registered with 1-2 mm accuracy. TRUS images are fused with subsequent TRUS, CT, and MRI images for diagnosis and treatment planning. This clinical work would be greatly enhanced if preceded by dual PET-TRUS exams that identified and localized the prostate cancerous tumors, rather than relying on only anatomical information.

### Example 1

### PET-TRUS Prostate Scanner and Imaging

LBNL Prostate-Optimized PET Scanner

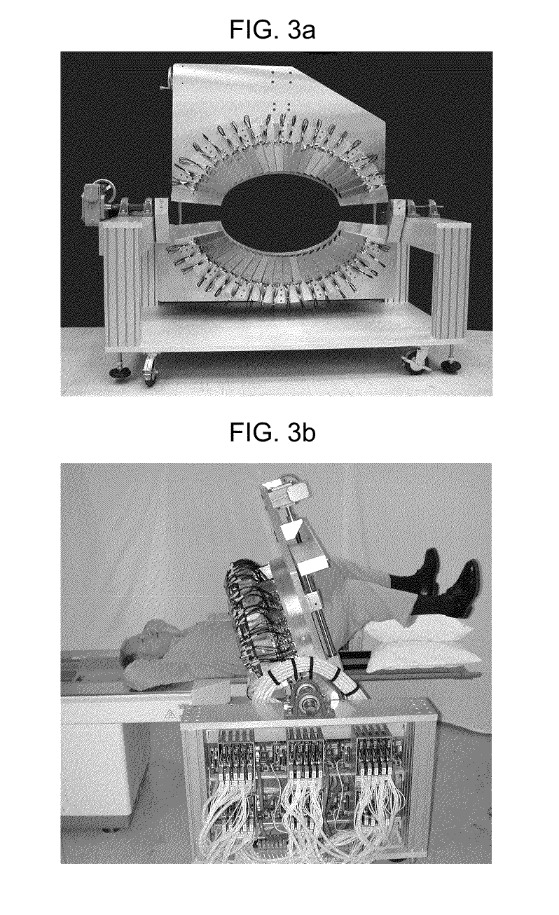

LBNL has built a high performance positron emission tomograph optimized to image the prostate [32-34]. Coincidence imaging of positron emitters is achieved using a pair of external curved detector banks with the patient centered between them. The two banks form an incomplete elliptical ring of detectors with a 45 cm minor axis and a 70 cm major axis, which reduces the distance between the detectors and patient. FIG. 3 shows the transaxial and sagittal views of the scanner. Each bank consists of two axial rows of 20 ECAT HR+PET block detector modules for a total of 80 detectors per scanner; thus the scanner uses about one-quarter the number of detectors as an EXACT HR or HR+ scanner. The ECAT HR+ block detectors are three attenuation lengths thick for good detection efficiency with narrow detector elements (i.e., 8×8 arrays of 4.4×4.1×30 mm³ BGO crystals) to achieve good spatial resolution. The individual detector modules are angled to point towards the scanner center (where the prostate will be positioned), thus reducing penetration effects for annihilation photons originating in the prostate region. We use modified front end, coincidence, and readout electronics originally developed by CTI Inc. for the HRRT PET scanner. Inter-module septa that extend 5 cm beyond the scintillator crystals reduce the background events from random coincidences and from photons that Compton scatter in the patient [J. Qi, J. S. Huber, R. H. Huesman, W. W. Moses, S. E. Derenzo, et al., “Septa Design for a Prostate Specific PET Camera,” IEEE Trans Nucl Sci, vol. NS-52, pp. 107-113, 2004.]. The scanner has a reduced axial extent (8 cm) and thus better shielding than a conventional whole body PET scanner, which reduces the number of scatter and random events. Hence, we achieve lower backgrounds and improved detection efficiency in the central imaging volume at a lower cost.

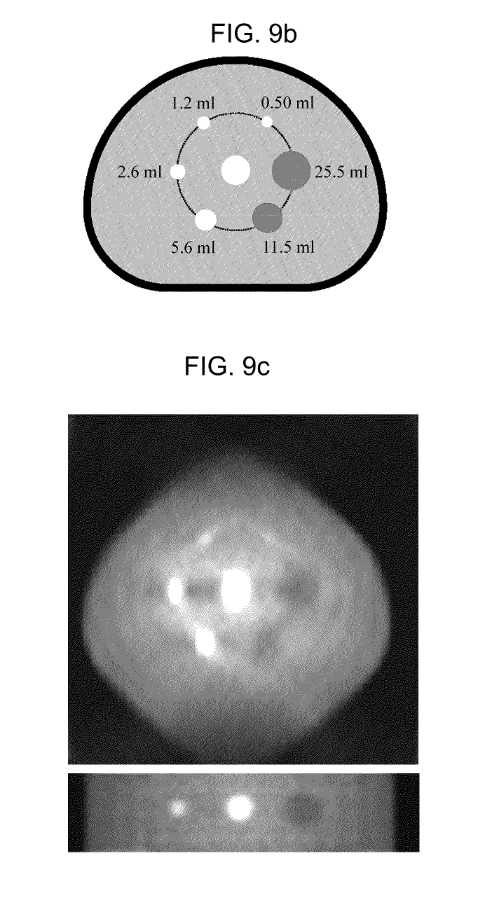

We use a 3D iterative penalized maximum likelihood reconstruction algorithm that is very flexible in modeling arbitrary scanner geometry [J. S. Huber, et al., IEEE Trans Nucl Sci, vol. NS-48, pp. 1506-1511, 2001; R. H. Huesman, et al., IEEE Trans Med Imag, vol. 19, pp. 532-537, 2000; and J. Hu, et al., Proceedings of The International Meeting on Fully Three-Dimensional Image Reconstruction in Radiology and Nuclear Medicine, pp. 416-420, Salt Lake City, Utah, 2005, hereby incorporated by reference]. We have characterized the completed scanner in 3D mode (i.e., without septa). The sensitivity of a point source in the center is 946 cps/μCi (2.6%). Using a 19 cm diameter cylinder phantom, the maximum total count rate is 528 kHz at 1.5 μCi/ml and the trues+scatter events cross the randoms at 0.41 μCi/ml. We have reconstructed images of line sources, and the spatial resolution is 4 mm full width at half maximum (FWHM) in the central region. We have also successfully reconstructed extended simple prostate and NEMA body phantoms [J. S. Huber, W. S. Choong, W. W. Moses, J. Qi, J. Hu, et al., “Initial Results of a Positron Tomograph for Prostate Imaging,” IEEE Trans Nucl Sci, NS-53, pp. 2653-2659 (2006)], as shown in FIG. 9.

Transrectal Ultrasound

Transrectal ultrasound imaging of the prostate is a standard imaging technique widely used for prostate cancer diagnosis, biopsy, treatment planning and brachytherapy seed placement. A volumetric 3D reconstructed image of the prostate can be generated using a series of 2D TRUS images. Such 3D images are currently used to determine the prostate volume and calculate dose for brachytherapy planning. The images are formed by mounting a transrectal probe to a fixture that is rigidly attached to the table through a calibrated linear stepper that allows displacement along its axis. Ultrasound images in the transverse plane (i.e., perpendicular to the probe axis) are acquired from base to apex. A complete 3D TRUS image of the prostate, urethra and rectum wall is then reconstructed using a series of 2D images taken in a step and shoot protocol. A physical puncture attachment for radiotherapeutic seed implantation is also attached to the probe fixture. A virtual grid position, corresponding to the projection of the puncture attachment holes, is projected on the image to provide localization. FIG. 4 shows a drawing of the TRUS unit with the probe inserted in a patient, as well as a 2D transverse ultrasound image with grid and the contours from a 3D reconstruction. We determine the position of the TRUS probe relative to the PET scanner, allowing the PET and TRUS images to be accurately co-registered using a simple rigid-body transformation specified by the alignment of the two systems.

### Example 2

### PET-TRUS Prostate Positioning

We used the LBNL prostate-optimized PET scanner, shown in FIG. 3, to acquire 3D volumetric PET images [J. S. Huber, W. S. Choong, W. W. Moses, J. Qi, J. Hu, et al., “Initial Results of a Positron Tomograph for Prostate Imaging,” IEEE Trans Nucl Sci, NS-53, pp. 2653-2659 (2006)]. We also use a commercial TRUS imaging system to acquire a series of 2D TRUS images, then reconstruct them to visualize a 3D TRUS image of the prostate region (see FIG. 4). We have mechanically modified this TRUS equipment to work when mounted onto a common patient table in conjunction with the PET scanner (see FIG. 5b). The TRUS probe is rigidly attached to the modified TRUS stepper that allows calibrated linear displacement along its axis. A point source holder (with two ⁶⁸Ge point sources) is attached to the TRUS stepper. The TRUS probe-stepper-point source holder unit is mounted onto a moveable TRUS stabilizer arm that is rigidly attached to the patient table. The stabilizer arm moves to allow correct positioning of the TRUS probe in a patient (or phantom), then its position is fixed by tightening a single knob.

Using this dual PET-TRUS system, we have developed a method to accurately position a prostate near the PET-center:
a. The TRUS probe is inserted inside a patient (or phantom), positioned at the prostate and the stabilizer arm is fixed.
b. A series of 2D TRUS prostate images in the transverse plane (i.e., perpendicular to the probe axis) is acquired from base to apex using the linear stepper.
c. The TRUS probe tip is positioned axially at the center of the prostate using the stepper.
d. The patient table is then moved so that two ⁶⁸Ge point sources are visually positioned near the PET-center with the aid of visible low-powered lasers. (These two ⁶⁸Ge point sources are placed in a holder, along the axial line of the TRUS probe, at a known location from the TRUS probe tip (FIG. 5b)).
e. PET data are acquired for 1-5 minutes, quickly reconstructed, and the location of the point sources determined in PET coordinates. We use a two-iteration expectation-maximization algorithm, with a simplified model of the PET scanner geometry, to quickly reconstruct the data in about 3 minutes. The resulting PET images are then quickly processed (within 1 minute) to determine the point source locations in PET coordinates, by determining the brightness-weighted average for each point source in the 3D volumetric PET image.
f. The two point source locations are represented by two position vectors and vector algebra is used to calculate the location of the TRUS probe tip (within 1 minute.)
g. The patient table is then moved to position the TRUS probe tip (i.e., prostate) at the PET-center.

Both our patient positioning and PET-TRUS image co-registration depend on our ability to determine the 3D location of the TRUS probe tip in PET coordinates (i.e., relative to the PET-center) using the method outlined above. However, these two tasks require different levels of accuracy. For patient positioning, a patient's prostate only needs to be positioned within the optimum central field of view of the PET scanner—within 3 cm from the PET-center in the transaxial plane and within 1 cm from the PET-center in the axial plane.

We have validated our ability to accurately determine the TRUS probe tip's 3D location in PET coordinates by imaging a point source that represents the prostate location. We attached a third 511 keV ⁶⁸Ge point source on the TRUS probe tip. We positioned the TRUS probe tip using the procedure described above, acquired (for 5 minutes) and reconstructed PET data, and determined the third point source location in PET coordinates. This procedure was repeated several times for each TRUS probe position. Two very different probe positions were measured—(1) with the probe roughly level to the patient table and pointing along the axial z-axis and (2) with the probe pointing down 11 degrees (i.e., at greater angle than expected for patients) and to the left 2 degrees. Although the spatial resolution for the PET scanner is 4 mm FWHM, the centroid of a point source profile can be more accurately determined. We were able to reproducibly position the TRUS probe tip within 1 mm from the PET-center in the axial direction (i.e., direction of patient table motion). Specifically, the third point source location on average was −0.64±0.08 mm from the PET-center in the axial direction. Thus, we have achieved greater accuracy than required for patient positioning.

We can also validate prostate positioning using a modified commercial TRUS prostate phantom (e.g., CIRS model 058), which is a clear acrylic box with structures simulating the prostate, rectal wall, seminal vesicles, urethra and perineal membrane (FIG. 7). Tubing is added at a fixed known location through the prostate of the TRUS phantom, and filled with 511 keV radioactive solution (e.g., ¹⁸F or ⁶⁸Ge water). The tubing material is chosen for clear ultrasound imaging (e.g., so the entire tube cross-section is visible in coronal ultrasound images) and appropriate dimensions (e.g., a large enough inner volume for the necessary 511 keV radioactive solution). For instance, silicone tubing (e.g. Tygon #3350 sanitary silicone tubing) with a 5/32″ inner diameter and 7/32″ outer diameter can be used. The phantom is placed on the patient table and imaged with the TRUS probe in 2-5 mm steps from base to apex of the prostate. The patient table is then moved to center the prostate at the PET-center (using the previously described procedure), PET phantom data is acquire and reconstructed. If the PET images indicate that the line source is near the PET-center, then prostate positioning is successfully validated. This can be further validated by repeating the phantom imaging procedure several times to confirm that the prostate positioning is reproducible.

### Example 3

### Co-Registration and PET-TRUS Prostate Phantoms

### Custom PET-US Phantom

We have developed a method to co-register PET and TRUS images and validate this method using a custom TRUS-PET prostate phantom. We construct and use a TRUS-PET prostate phantom with structures that simulate the acoustical properties for TRUS and 511 keV activity concentrations for PET. We use agar-gelatin-based tissue mimicking materials (TMMs) that are mixed with radioactive water solutions [J. S. Huber, Q. Peng, and W. W. Moses, “Multi-Modality Phantom Development,” IEEE Nuclear Science Symposium Conference Record 2007, vol. 4, pp. 2944-2948, (Edited by B. Yu), Honolulu, Hi., 2007]. When developing the procedures for the agar-gelatin-based phantom construction, we first used non-radioactive water to test ultrasound properties. We then used short-lived ¹⁸F radioactive (110 minutes half-life) water solutions, since ¹⁸F is readily available from our in-house cyclotron and no radioactive waste is generated by these construction tests (since ¹⁸F quickly decays away). We will also use long-lived ⁶⁸Ge radioactivity (271 days half-life) for phantom construction to allow repeated PET imaging of the same phantom over at least one year.

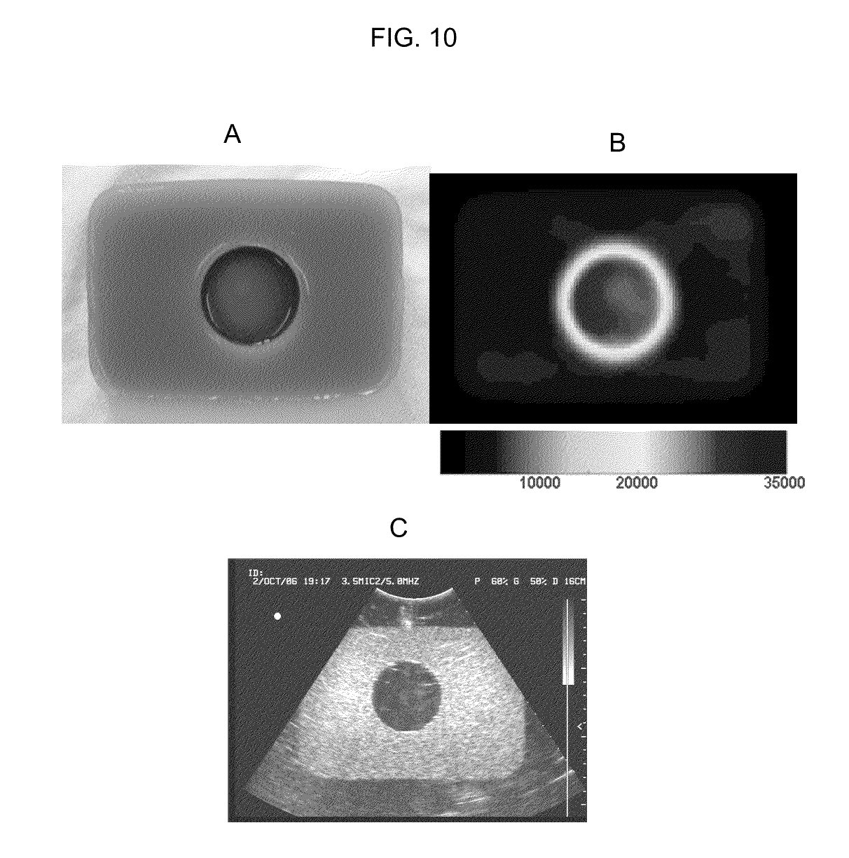

We have constructed a simple PET-ultrasound prostate phantom as proof of principle. We are not aware of previous work on the manufacturing of PET-ultrasound phantoms. Our simple PET-ultrasound phantom was constructed in two stages. We first filled a rectangular plastic box with 4% agarose that was prepared as a high-scatter ultrasound tissue mimicking material (TMM), creating an inclusion with a petrolatum-coated plastic rod in the center. Once the “pelvis” agarose hardened, the rod was removed and we filled the inner cylindrical “prostate” region with a low-scatter 8% gelatin TMM. FIG. 10a shows a photograph of the final phantom. At each stage, the TMM was mixed with ¹⁸F radioactive (110 minute half-life) water solution before putting it into a refrigerator to harden. The phantom had six times higher 511 keV activity density in the inner cylinder “prostate” than in the outer “pelvis.” It was roughly centered in an EXACT HR PET scanner, and PET data were acquired with a 10 minute transmission scan followed by a 20 minute emission scan in 3D mode. At the start of the emission scan, the ¹⁸F activity density was 1.07 μCi/ml in the inner cylinder gelatin and 0.17 μCi/ml in the background agarose. Reconstruction was performed with attenuation and scatter correction. FIG. 10b shows a reconstructed coronal PET image of the phantom. The ¹⁸F activity uniformly concentrated in the cylindrical “prostate” gelatin is clearly visible within the “pelvis” background activity. The phantom was then imaged using a 5 MHz external Elektra ultrasound system, as shown in FIG. 10c. The ultrasound image clearly shows the low-scatter cylindrical “prostate” gelatin, which is surrounded by the high-scatter “pelvis” agarose. Thus, we have demonstrated our ability to construct and image a custom PET-ultrasound phantom. However, the phantom's mechanical and ultrasound properties did not have long-term stability especially at room temperature.

### Custom PET-US-CT-MRI Phantom

Since the custom PET-US phantom described above did not have long-term stability at room temperature, we also developed a different phantom construction process. We have constructed a multi-modality phantom using tissue mimicking mixtures of agar, gelatin, CuCl₂-2H₂O, EDTA-tetra Na Hydrate, NaCl, HCHO, Germall-Plus™, glass beads, BaSO₄, and deionized water (Table I). Similar agar-gelatin mixtures were proven to have long-term mechanical, ultrasound and MRI properties for at least one year [E. L. Madsen, M. A. Hobson, S. Hairong, T. Varghese and G. R. Frank, “Tissue-mimicking agar/gelatin material for use in heterogeneous elastography phantoms,” Phys. Med. Biol., vol. 50, pp. 5597-5618, 2005]. These agar-gelatin-based tissue mimicking materials can be mixed with radioactive water solutions. When developing the procedures for the agar-gelatin-based phantom construction, we used non-radioactive water and short-lived ¹⁸F radioactive water solutions. We can use long-lived ⁶⁸Ge radioactivity (271 day half-life) for phantom construction to allow repeated PET imaging of the same phantom over at least one year.

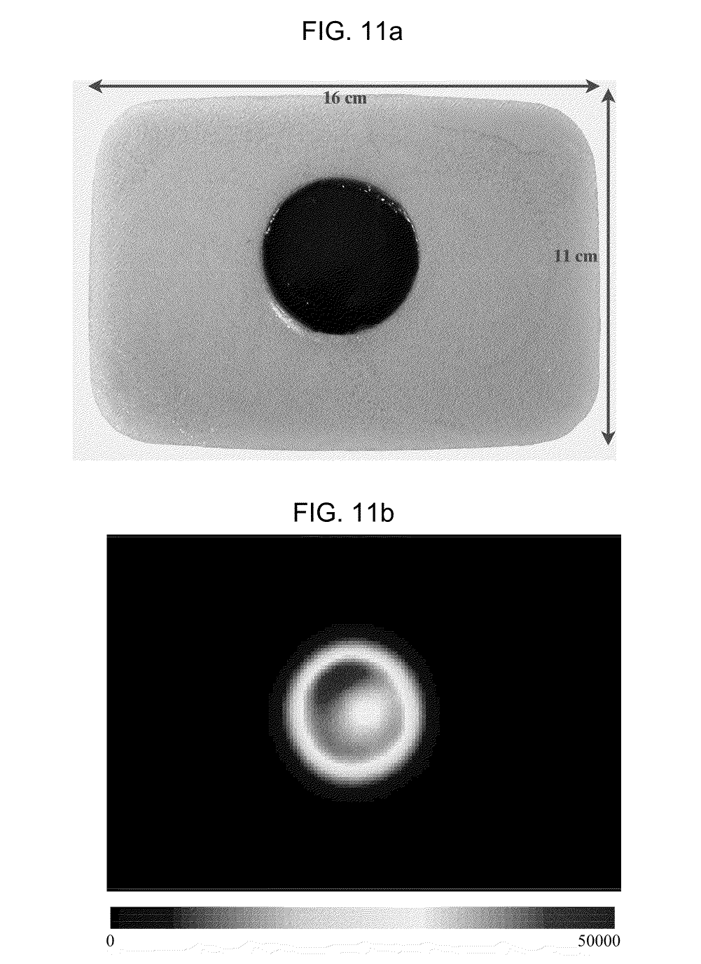

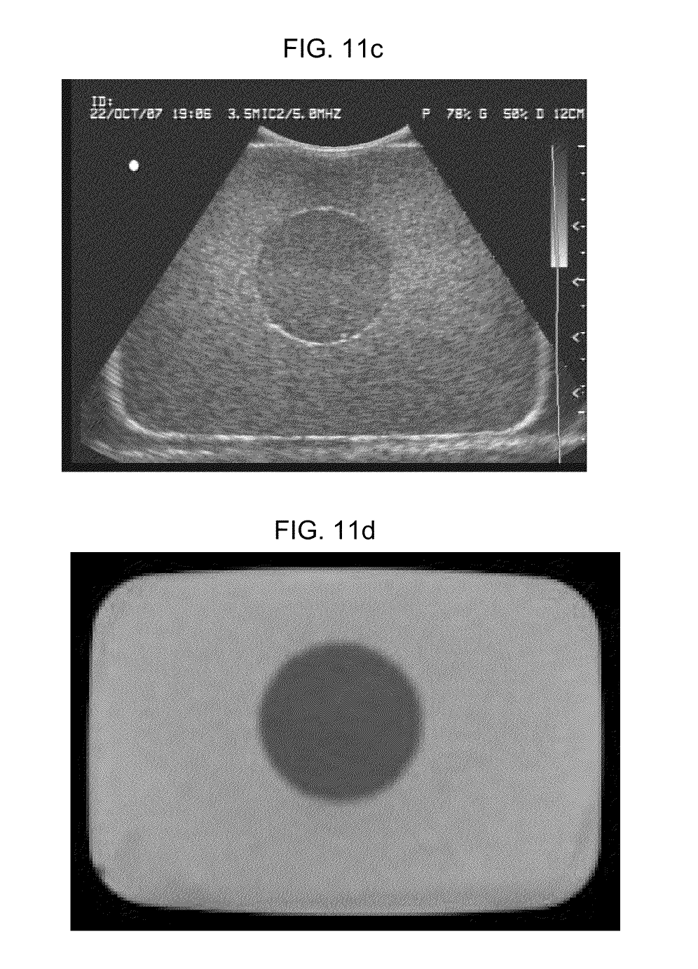

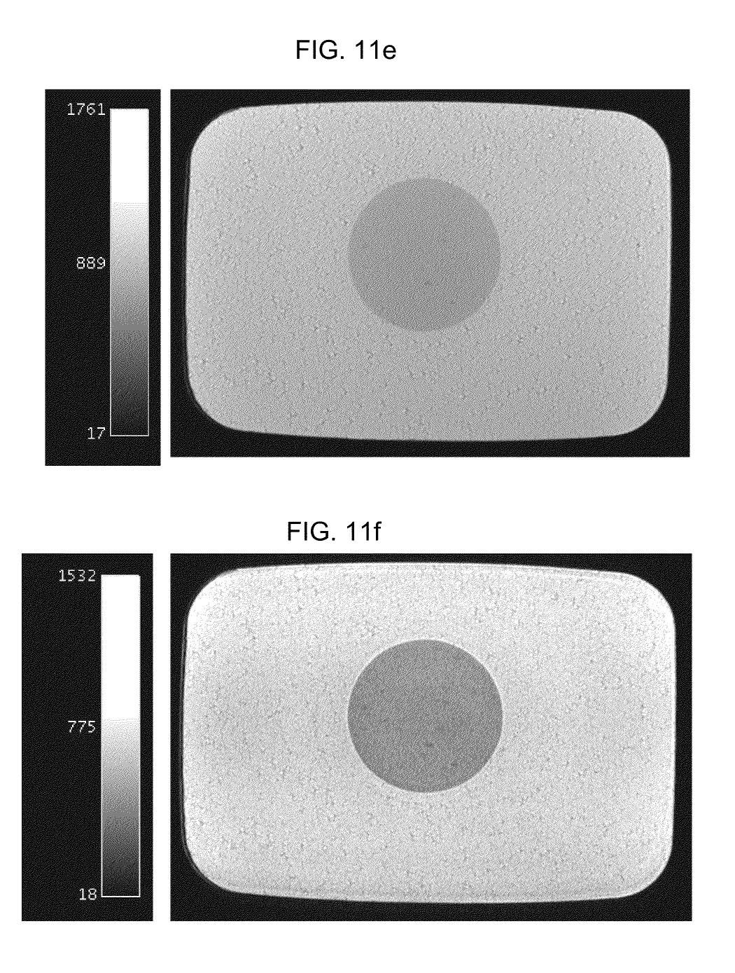

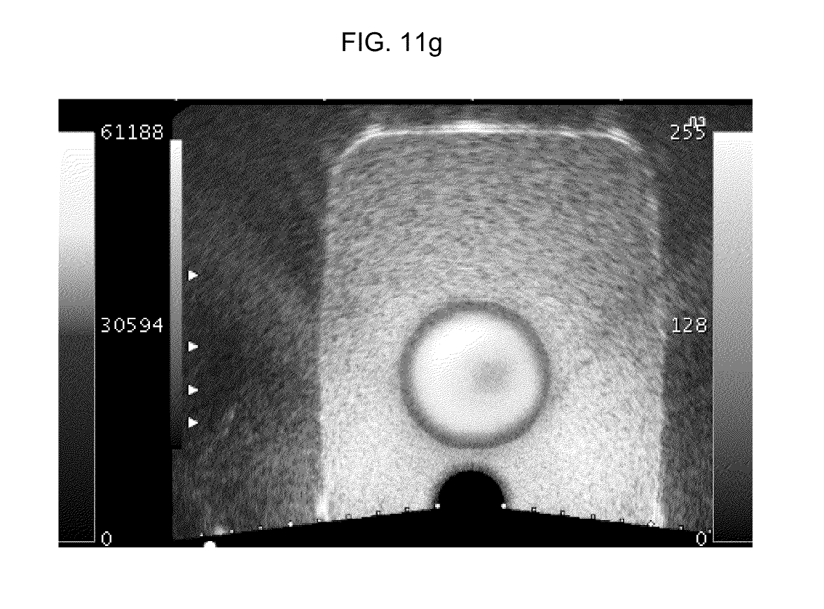

We constructed a two-region PET-US-CT-MRI phantom with an inner cylindrical “prostate” within an outer rectangular “pelvis” (i.e., with the same simple geometry as the PET-US phantom described above). We first filled the rectangular “pelvis” container with the “Pelvis TMM” (Table I), creating a void with a petrolatum-coated plastic rod in the center. This rod was removed once the rectangular “pelvis” hardened, then we filled the inner cylindrical “prostate” with a “Prostate TMM” (Table I) having different multi-modality properties and ¹⁸F activity. The TMMs were hardened at room temperature. FIG. 11a shows a photograph of the custom PET-US-CT-MRI phantom.

TABLE I
Dry-weight percents of the various components in the PET-US-CT-MRI custom phantom.

| | Agar | Gelatin | CuCl₂—2H₂O | EDTA | NaCl | HCHO | Germall-Plus | Glass Beads | BaSO₄ |
|---|---|---|---|---|---|---|---|---|---|
| Pelvis TMM | 1.17 | 5.50 | 0.11 | 0.32 | 0.77 | 0.24 | 1.44 | 4.38 | 0.50 |
| Prostate TMM | 3.64 | 5.70 | 0.12 | 0.34 | 0.80 | 0.25 | 1.50 | 0 | 0 |

The weight percent of the water (mixed with a small volume of radioactive solution) is not shown since it makes up the remainder.

Table I outlines the dry-weight percents for the “Prostate TMM” and “Pelvis TMM” used to construct the custom PET-US-CT-MRI phantom. For these tests, only the cylindrical “prostate” TMM was mixed with a ¹⁸F-water solution. The primary role of each ingredient is summarized below:

Agar: concentration set to achieve tissue-like US properties, such as US propagation speed. Higher agar concentration also produces shorter longitudinal (T₁) and transverse (T₂) MRI relaxation times.

Gelatin: concentration set for tissue-like US properties, such as US propagation speed.

Concentration must be roughly the same for “prostate” and “pelvis” regions to avoid changes in volumes due to osmosis.

CuCl₂-2H₂O and EDTA-tetra Na Hydrate: EDTA forms chelate with the Cu²⁺ ions to allow Cu²⁺ to remain mobile, allowing controlled lowering of the T₁ MRI relaxation time.

NaCl: anti-bacterial agent that produces tissue-like MRI coil loading.

HCHO (37% formaldehyde): cross-links the gelatin, raising the melting point to 78° C. where the agar component melts.

Germall-Plus™: preservative to prevent fungal and bacterial invasion.

Glass Beads (20 μm average diameter): increases ultrasound attenuation and backscatter to tissue-like levels. Also lowers T₁ and T₂ MRI relaxation times.

BaSO₄: increases radiographic attenuation for CT.

¹⁸F: 511 keV radioactivity for PET imaging.

The expected ultrasound properties include a propagation speed of about 1535 m/s, a density of about 1.04 g/ml, and an attenuation coefficient divided by frequency of about 0.14 dB/cm/MHz for the “Prostate TMM” and 0.38 dB/cm/MHz for the “Pelvis TMM.” The MRI T₁ relaxation times are expected to be about 494 ms for the “Prostate TMM” and 423 ms for the “Pelvis TMM” [E. L. Madsen, M. A. Hobson, S. Hairong, T. Varghese and G. R. Frank, “Tissue-mimicking agar/gelatin material for use in heterogeneous elastography phantoms,” Phys. Med. Biol., vol. 50, pp. 5597-5618, 2005].

The custom PET-US-CT-MRI phantom was imaged using an EXACT HR PET scanner. PET data were acquired with a 3D emission scan followed by a 10 minute transmission scan. Iterative image reconstruction was performed with attenuation and scatter correction. FIG. 11b shows a reconstructed coronal PET image of the phantom. The ¹⁸F activity concentrated in the cylindrical “prostate” is clearly visible. The PET image in the “prostate” region is not uniform in this case (e.g., compared to FIG. 10b) due to PET scanner hardware problems that affected the attenuation map and normalization.

The custom PET-US-CT-MRI phantom was imaged by the other three modalities the following day, after the ¹⁸F radioactivity decayed. The phantom was imaged with a 5 MHz external Elektra ultrasound system. FIG. 11c shows an ultrasound image of the phantom with a lower-scatter cylindrical “prostate” surrounded by higher-scatter “pelvis.” The phantom was then imaged with a Hawkeye CT scanner (140 keV; 2.5 mAmps) on a Millennium VG3 SPECT gantry. Reconstruction was performed with filtered backprojection using a Hann filter with a cutoff frequency of 1. FIG. 11d shows a reconstructed coronal CT image of the phantom with increased radiographic attenuation in the “pelvis” due to the BaSO₄.

The phantom was then imaged with an 1.5 T Avanto Siemens MRI scanner using a head coil with a T₁-weighted 2D spin echo pulse sequence (TE=7.8 msec; TR=500 msec; field of view=220 mm×178.8 mm×3 mm; voxel size=0.4 mm×0.4 mm×3 mm) FIG. 11e shows a reconstructed T₁-weighted MRI image with a darker “prostate” representing a longer T₁ compared to the “pelvis.” The glass beads (used for ultrasound imaging) shortened the T₁ in the “pelvis” to make it brighter, despite the increased agar concentration in the “prostate.” The phantom was also imaged with a T₂-weighted 2D turbo spin echo pulse sequence (TE=91 msec; TR=4000 msec; field of view=220 mm×175 3 mm×3 mm; voxel size=0.4 mm×0.4 mm×3 mm). FIG. 11f shows a reconstructed T₂-weighted MRI image with a darker “prostate” representing a shorter T₂ compared to the “pelvis.

Finally, we removed a cylindrical section of the gel in the pelvis region, in order to allow transrectal ultrasound imaging. Transrectal ultrasound imaging was performed using a Hitachi Hi Vision 5500 system with a EUP-U533 biplane transrectal ultrasound probe. FIG. 11g shows a fused PET (color) and TRUS (grayscale) coronal image of the phantom. The OsiriX software package was used for the image fusion.

We plan to make a PET-TRUS prostate phantom with a more realistic geometry using ⁶⁸Ge water in our agar-gelatin mixture. The phantom will have structures simulating the prostate, rectum and rectal wall and urethra in a background gel with an opening for the TRUS probe (FIG. 8). The urethra is routinely simulated by filling a tube with ultrasound gel with some air bubbles. We will image this PET-TRUS prostate phantom with PET and TRUS to confirm that we have produced a phantom with the required properties. Since this PET-TRUS prostate phantom will be used only to validate image co-registration, the phantom does not have to exactly mimic the PET and TRUS properties of the prostate region.

A custom PET-TRUS-CT-MRI phantom using ⁶⁸GeCl₄ in a 0.5M HCl solution was also constructed. The dry-weight percents of the various components of the custom PET-TRUS-CT-MRI are shown in Table 2 below. The remaining weight percent is deionized water (mixed with a small amount of radioactive solution).

TABLE 2
Dry-weight percents of components for custom PET-TRUS-CT-MRI phantom.

| | Agar | Gelatin | CuCl₂—2H₂O | EDTA | NaCl | HCHO | Germall-Plus | Glass Beads | BaSO₄ |
|---|---|---|---|---|---|---|---|---|---|
| Pelvis TMM | 1.17 | 5.52 | 0.11 | 0.33 | 0.77 | 0.24 | 1.45 | 4.4 | 0.50 |
| Prostate TMM | 3.64 | 5.70 | 0.12 | 0.34 | 0.80 | 0.25 | 1.50 | 0 | 0 |

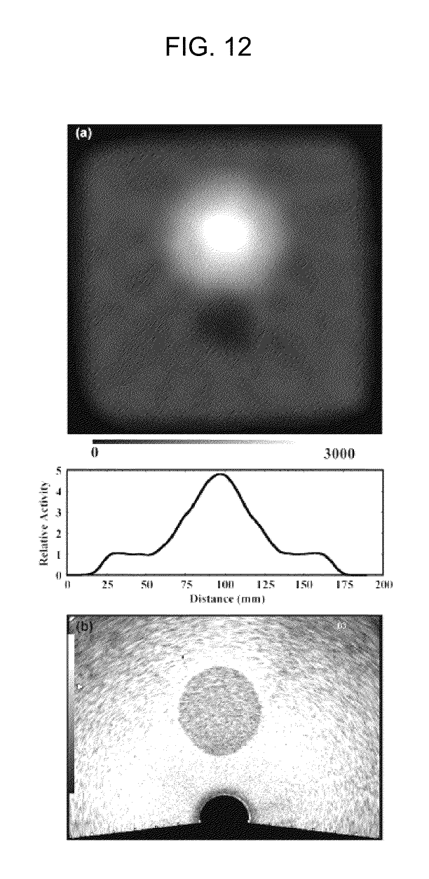

The phantom gel is inside a plastic cubic box with a lid that has a hole for TRUS imaging access. In order to prevent ⁶⁸Ge-gel pieces from escaping during TRUS imaging, a condom is used to seal the hole. (The condom base is fixed around a centering ring on top of the container lid. The condom is held in place using a small washer inside the condom tip and a magnet outside the bottom of the plastic container.) At the time of the phantom construction, the ⁶⁸Ge activity density was 0.73 μCi/ml in the “prostate” and 0.12 μCi/ml in the “pelvis.” This custom phantom was imaged using an EXACT HR PET scanner 30 hours after phantom construction. PET data were acquired with a 3D emission scan followed by a 10 minute transmission scan. Iterative image reconstruction was performed with attenuation and scatter correction. FIG. 12a shows a reconstructed coronal PET image of the phantom, as well as a horizontal profile through the “prostate” center. The ⁶⁸Ge activity density was six times higher in the “prostate” than the “pelvis.” The ⁶⁸Ge activity in the “prostate” is clearly visible within the “pelvis” background. However, the “prostate” radioactivity in the PET image has a blurrier edge in this case, and the relative activity shown in the profile is only 4.8 (“prostate”) to 1 (“pelvis”). This is probably due to the initial migration of the ⁶⁸Ge tetrachloride molecules, which is discussed in detail below. This assumption is supported by the uniform radioactivity seen in the PET image of a comparable PET-TRUS-CT-MRI phantom constructed with ¹⁸F water. The phantom was also imaged with a Hitachi Hi-Vision 5500 digital ultrasound system, using a B mode bi-plane TRUS probe in a linear stepper. FIG. 12b shows a transrectal ultrasound image of the phantom with a lower-scatter“prostate” surrounded by a higher-scatter “pelvis.”

We intended to use this custom PET-TRUS-CT-MRI phantom repeatedly over a year. However, the ⁶⁸Ge tetrachloride molecules in the “prostate” migrated into the “pelvis” to become roughly uniformly distributed throughout the phantom in less than 57 days. We believe that the ⁶⁸Ge tetrachloride molecules were small enough to penetrate the gel pores, slowly reaching an equilibrium in radioactive concentration throughout the “prostate” and “pelvis.” We should be able to prevent this ⁶⁸GeCl₄ diffusion by using a barrier, such as a female latex condom, between the “prostate” and “pelvis” so the radioactivity instead reaches a uniform equilibrium within the “prostate” and “pelvis” separately.

The phantom can be constructed with structures simulating male and/or female anatomical organs, including but not limited to, prostate, rectum, rectal wall, urethra, ovaries, uterus, etc.

### Example 4

### Co-Registration and PET-TRUS Prostate Patient Imaging

Dual PET-TRUS imaging will also be validated with patient studies. The patients will have prostate cancer that has been confirmed by biopsy, and most patients will be imaged prior to any treatment. The dual PET-TRUS studies will be performed at the Medical Imaging Technology Department at LBNL in building 55 using the LBNL prostate-optimized PET scanner (or the Siemens ECAT EXACT HR PET scanner, if needed as backup) with a commercial transrectal ultrasound system (e.g., Hitachi Hi Vision 5500 with a EUP-U533 bi-plane transrectal transducer probe, Accucare EXIT stepper, and micro-touch stabilizer arm, or equivalent). Clinicians will administer consent, and will be available during the entire dual PET-TRUS procedure. Patients who are scheduled must meet protocol inclusion/exclusion criteria, and these will be reviewed by the attending physician prior to the procedure. During the clinician-patient conference, a short history of the present disease and an accounting of recent (48 hours) food ingestion will be recorded. The dietary information is important because choline biodistribution results might be affected by recent food ingestion.

Patients will be asked to be on a light diet one day prior to this study, following a clear liquid only diet the previous day and eating nothing after midnight the night prior to the procedure. Patients will empty their bladder 30 minutes or more before the procedure, allowing for some residual liquid to be present in the bladder during the PET-TRUS scan. Patients will empty their rectum prior to the procedure, using an enema only as needed. Although enema bowel prep is common for TRUS-guided biopsy, an enema is rarely necessary for a standalone transrectal ultrasound procedure. If necessary, patients will perform a self-administered Fleets enema about 10 minutes prior to the PET-TRUS scan. (Most patients can easily self administer the enema in about 5 minutes by bending over at the waist, inserting and injecting the enema, and evacuating into the toilet.)

Patients will be comfortably positioned on the patient table, lying on their back with their legs raised, for the dual PET-TRUS scan. Pillows and knee supports will be used for patient comfort. The clinician will insert the commercial transrectal probe (with condom) and position it at the prostate. A series of 2D ultrasound images will be acquired in the transverse plane from prostate base to apex (about 25 slices or less). No anesthesia will be used, in accordance with standard clinical practice. The TRUS probe tip will then be positioned axially at the center of the prostate using the stepper.

Leaving the transrectal probe in place, the patient table will be moved a short distance at a slow speed to position two low-activity (<50μCi) Ge-68 point sources near the center of the PET scanner with the aid of visible low-powered lasers (which define the major and minor axis of the PET scanner). These Ge-68 point sources are placed in a holder along the axial line of the TRUS probe at a known distance from the TRUS probe tip. PET data from these Ge-68 point sources will be quickly acquired and analyzed to determine the current location of the TRUS probe tip (which is positioned axially at the center of the patient's prostate). These sources will expose the patient to a small dose because: they are NOT inserted into the patient; they are placed at least 2 inches from the patient; and they will be near the patient for a short time. Leaving the transrectal probe in place, the patient table will be moved a short distance at a slow speed to position the patient's prostate near the PET-center in preparation for PET imaging.

A clinician will insert a small intravenous catheter and then immediately administer the PET radiopharmaceutical through the catheter. He will inject up to 10 mCi (±10%) of [¹¹C]choline per patient. PET imaging over the prostate and neighboring regions will commence immediately following injection and proceed for up to 40 minutes. No transmission scans will be performed with the LBNL prostate-optimized PET scanner. No blood sampling will be necessary (arterial or venous) for this procedure. Patients will be taken to the bathroom to urinate immediately following their dual PET-TRUS scan. The total time of the dual PET-TRUS procedure will be less than an hour, so the transrectal probe will be left in place for up to an hour.

PET images will be reconstructed using a 3D iterative penalized maximum likelihood algorithm as described in in J. S. Huber, S. E. Derenzo, J. Qi, W. W. Moses, R. H. Huesman, et al., “Conceptual Design of a Compact Positron Tomograph for Prostate Imaging,” IEEE Trans Nucl Sci, vol. NS-48, pp. 1506-1511, 2001; R. H. Huesman, G. J. Klein, W. W. Moses, J. Qi, B. W. Reutter, et al., “List mode maximum likelihood reconstruction applied to positron emission mammography with irregular sampling.,” IEEE Trans Med Imag, vol. 19, pp. 532-537, 2000; and J. Hu, J. Qi, J. S. Huber, W. W. Moses and R. H. Huesman, “MAP image reconstruction for arbitrary geometry PET systems with application to a prostate-specific scanner.” Proceedings of The International Meeting on Fully Three-Dimensional Image Reconstruction in Radiology and Nuclear Medicine, pp. 416-420, Salt Lake City, Utah, 2005, if the PET scanner used is the prostate-optimized PET scanner. The attenuation correction factors will be calculated based on body contours and a uniform attenuation coefficient. Anatomical boundaries will be obtained from the outer edges of emission sinograms acquired from transverse sections [C. Michel, A. Bol, A. G. DeVolder and A. M. Goffinet, “Online brain attenuation correction in PET: towards a fully automated data handling in a clinical environment,” Euro J Nucl Med, vol. 15, pp. 712-718, 1989]. Similarly, attenuation correction will be made for the TRUS probe (which is left in place at a known location during the PET scan) and the patient table. 3D anatomy contours will be identified from the TRUS images, and these TRUS contours will be superimposed onto the corresponding (resliced) PET images for anatomical localization [G. J. Klein, X. Teng, W. J. Jagust, J. L. Eberling, A. Acharya, et al., “A methodology for specifying PET VOI's using multimodality techniques,” IEEE Trans Med Imag, vol. 16, pp. 405-415, 1997, R. H. Huesman, G. J. Klein, J. A. Kimdon, C. Kuo and S. Majumdar, “Deformable registration of multimodal data including rigid structures,” IEEE Trans Nucl Sci, vol. 50, pp. 389-392, 2003].

Since the patient is known to have prostate cancer, the [¹¹C]choline uptake in his prostate should be visible with the PET scanner within 3 minutes after injection if the patient is positioned properly. The patient positioning technique is successfully validated if the prostate has been positioned within the optimum central field of view of the PET scanner—within 3 cm from the PET-center in the transaxial plane and 1 cm from the PET-center in the axial direction—as evidenced by PET imaging. If no [¹¹C]choline uptake is seen in the field of view of the PET scanner, then there is some uncertainty. This patient may not have enhanced [¹¹C]choline uptake in his prostate or he may not have been properly positioned in the PET scanner. Further PET data will be acquired once the initial 15 minutes are complete. The patient table will be moved ±7 cm from the initial position and PET data will be acquired for 5 additional minutes at each position. If the patient has no enhanced [¹¹C]choline uptake in either of these neighboring regions, then the patient positioning will still be considered valid.

The total time of the PET-TRUS study will typically be 40 minutes: 2 minutes to insert and position the TRUS probe in the patient, 3 minutes for TRUS data acquisition, 10 minutes to position the patient's prostate near the PET-center (using point sources on stepper), 15 minutes for [¹¹C]choline injection and PET data acquisition with the patient centered in the PET scanner, and 10 minutes to acquire PET data at the neighboring regions. The total time commitment for the study, including patient consent, will be approximately 2 hours.

Patients will be recruited and after clinical evaluations, patients deemed appropriate for inclusion will be referred for study. Patients will have confirmed prostate cancer and will not have received treatment in the preceding four weeks, allowing time for initial healing to occur in order to minimize [¹¹C]choline accumulation in inflammatory tissue.

The transrectal probe will be covered with a new condom prior to each use. Following standard clinical cleaning practices, the ultrasound equipment will be brushed with disinfectant and washed with alcohol after each use. No follow-up visits are planned. However, the patient's prostate cancer status will be followed over a period of 3 years to provide a more accurate and thorough comparison between (a) the location of abnormal [¹¹C]choline uptake seen on the dual PET-TRUS scan and (b) the location of histologically confirmed prostate cancer. For instance, dual PET-TRUS prostate imaging could accurately detect abnormal uptake in a patient's prostate region before conventional clinical procedures (e.g., biopsy) are able to detect the prostate cancer. Following the patient's prostate cancer status will allow us to evaluate discrepancies of this kind, determining if the location of abnormal radiotracer uptake accurately reflects the location of prostate cancer, so we are able to correctly evaluate our development of dual PET-TRUS prostate imaging. Hence, both current data and subsequent data relevant to the patient's prostate cancer status over a period of 3 years will be accessed in accordance with HIPAA regulations.

The above examples are provided to illustrate the invention but not to limit its scope. Other variants of the invention will be readily apparent to one of ordinary skill in the art and are encompassed by the appended claims. All publications, databases, and patents cited herein are hereby incorporated by reference for all purposes.

What is claimed is:

1. A method for accurate co-registration for dual-modality positron emission tomography (PET) and transrectal ultrasound (TRUS) imaging for prostate cancer, comprising the steps of:
a. providing a PET-TRUS system comprising a PET scanner having a patient table with a TRUS probe attached to the table through a TRUS calibrated linear stepper-arm assembly, wherein a holder with 511 keV point sources is mounted onto the TRUS stepper or probe;
b. inserting the TRUS probe inside the rectum of a patient, positioning said TRUS probe at the prostate, and fixing the stabilizer arm;
c. acquiring TRUS prostate images of the entire prostate region;
d. positioning the TRUS probe tip axially at the center of the prostate using the stepper;
e. moving the patient table so that at least two ⁶⁸Ge point sources are visually positioned near the PET-center with the aid of visible low-powered lasers;
f. acquiring and analyzing PET point source data, and determining the location of the point sources in PET coordinates;
g. calculating the location of the TRUS probe tip;
h. moving the patient table to position the TRUS probe tip at the PET-center;
i. injecting a 511 keV radiopharmaceutical into the patient and acquiring PET image data of the patient;
j. contouring the TRUS data two-dimensionally and reconstructing a three-dimensional TRUS image of the prostate region;
k. reconstructing three-dimensionally the PET data; and
l. accurately superimposing the PET and TRUS images, thereby resulting in a PET-TRUS image of the patient's prostate region showing anatomical and functional detail for precise localization of any prostate cancer.

2. The method of claim 1, wherein 511 keV point sources are placed on TRUS equipment and used to determine the location of the TRUS probe relative to the PET-center.

3. The method of claim 1, wherein steps (f)-(g), and (j)-(l) are carried out using computer executable logic.

4. A method of validating claim 1, wherein a custom PET-ultrasound phantom is constructed and imaged using a dual PET-TRUS system instead of a patient.

5. A multi-modality pelvic phantom comprising a shaped gel structure comprising an inner cylindrical or spherical pelvic region within an outer rectangular pelvic region formed by agar-gelatin-based tissue mimicking mixtures and radioactive solutions compatible with multiple imaging modalities.

6. The multi-modality pelvic phantom of claim 5, further comprising structures simulating the rectum, rectal wall and urethra in a background gel with an opening for insertion of a TRUS probe in the rectum.

7. The multi-modality pelvic phantom of claim 5, further comprising structures simulating other anatomical organs in the pelvic region.

8. The multi-modality pelvic phantom of claim 5, wherein the tissue mimicking mixture comprising agar, gelatin, CuCl₂-2H₂O, EDTA-tetra Na Hydrate, NaCl, HCHO, anti-bacterial and/or anti-fungal preservative, glass beads, BaSO₄, and radioactive solutions.

9. The multi-modality pelvic phantom of claim 5, wherein the tissue mimicking mixture forming the inner and outer pelvic regions comprising agar, gelatin, CuCl₂-2H₂O, EDTA-tetra Na Hydrate, NaCl, HCHO, anti-bacterial and/or anti-fungal preservative, glass beads, BaSO₄, and radioactive solution as set forth in the following Table :

| | Agar | Gelatin | CuCl₂—2H₂O | EDTA | NaCl | HCHO | Germall-Plus | Glass Beads | BaSO₄ |
|---|---|---|---|---|---|---|---|---|---|
| Pelvis TMM | 1.17 | 5.50 | 0.11 | 0.32 | 0.77 | 0.24 | 1.44 | 4.38 | 0.50 |
| Prostate TMM | 3.64 | 5.70 | 0.12 | 0.34 | 0.80 | 0.25 | 1.50 | 0 | 0 |

wherein the remaining dry-weight percentage comprising deionized water and radioactive solution.

10. The multi-modality pelvic phantom of claim 5, wherein the multiple imaging modalities are positron emission tomography (PET) and ultrasound (US).

11. The multi-modality pelvic phantom of claim 10, wherein the multi-modality phantom is further compatible with magnetic resonance (MR) and/or composite tomography (CT) imaging.

12. The multi-modality pelvic phantom of claim 5, wherein the multiple imaging modalities are positron emission tomography (PET) and magnetic resonance (MR) imaging.

13. The multi-modality pelvic phantom of claim 5, further comprising a barrier between the inner and outer pelvic regions.

14. A PET-TRUS-CT-MRI multi-modality phantom comprising a shaped gel structure with two pelvic regions and housed in a rigid container, wherein the the two pelvic regions are distinguishable by all four imaging modalities, wherein the inner pelvic region and outer pelvic region are formed by agar-gelatin-based tissue mimicking mixtures and radioactive solutions.

15. The multi-modality phantom of claim 14, wherein the agar-gelatin-based tissue mimicking mixtures and radioactive solutions comprising the dry-weight percentages as shown in the following Table:

| | Agar | Gelatin | CuCl₂—2H₂O | EDTA | NaCl | HCHO | Germall-Plus | Glass Beads | BaSO₄ |
|---|---|---|---|---|---|---|---|---|---|
| Pelvis TMM | 1.17 | 5.52 | 0.11 | 0.32 | 0.77 | 0.24 | 1.45 | 4.4 | 0.50 |
| Prostate TMM | 3.64 | 5.70 | 0.12 | 0.34 | 0.80 | 0.25 | 1.50 | 0 | 0 |

wherein the remaining dry-weight percentage comprising deionized water and radioactive solution, and wherein said multi-modality phantom has long-term stability of over six months at room temperature.

16. The multi-modality phantom of claim 14, further comprising a barrier between the inner and outer pelvic regions.
